(19) United States
(12) Patent Application Publication
(10) Pub. No.: US 2021/0353767 A1
(43) Pub. Date: Nov. 18, 2021

(54) COMPOSITIONS AND METHODS FOR DRUG DELIVERY

(71) Applicant: NORTH CAROLINA STATE UNIVERSITY, Raleigh, NC (US)

(72) Inventors: Yevgeny BRUDNO, Cary, NC (US); Mary R. Adams, Raleigh, NC (US)

(21) Appl. No.: 17/281,025

(22) PCT Filed: Sep. 30, 2019
(86) PCT No.: PCT/US2019/053734

§ 371 Date: Mar. 29, 2021

Related U.S. Application Data
(60) Provisional application No. 62/738,564, filed on Sep. 28, 2018.

(51) Int. Cl. A61K 47/68; A61P 35/00

(52) U.S. Cl. CPC A61K 47/6889; A61P 35/00

(57) ABSTRACT

Disclosed herein are methods for drug delivery, as well as kits for drug delivery.

### CROSS REFERENCE TO RELATED APPLICATIONS

This application claims the benefit of U.S. Provisional Application No. 62/738,564, filed Sep. 28, 2018, which is incorporated herein by reference in its entirety.

### BACKGROUND

Drug-eluting polymer systems (depots) have proven useful in a variety of clinical settings, including prevention of restenosis with stenting, cancer treatment and enhancing wound healing. These systems benefit from tunable drug release kinetics, days or even weeks of continuous drug release, and local delivery which together provide spatiotemporal control over drug availability and can diminish drug toxicity. However, existing drug-eluting systems have a finite depot of drug and become unneeded when spent and, in the case of non-degrading systems, may need surgical removal. For many therapeutic applications, an invasive procedure is needed to inject or implant a drug-eluting device, and these devices cannot be refilled or replaced without another invasive surgery. Thus, there exists an ongoing and unmet need for new and less-invasive methods for drug delivery.

### SUMMARY

Disclosed are methods for delivering an active agent to a target tissue in a subject. The methods can comprise contacting the target tissue with a Click Target defined by Formula I

X-L¹-CM¹ Formula I

wherein X represents a tissue binding moiety; L¹ is absent, or represents a linking group; and CM¹ represents a first click motif; and contacting the target tissue with a Click Prodrug defined by Formula II

A-L²-CM² Formula II

wherein A represents an active agent; L¹ is absent, or represents a linking group; and CM² represents a second click motif complementary to the first click motif.

In some embodiments, the tissue binding moiety can comprise a functional group capable of chemically reacting with a functional group in a peptide to form a covalent bond. In certain embodiments, the tissue binding moiety comprises a functional group capable of chemically reacting with an amine group in a peptide to form a covalent bond, such as a sulfo-hydroxysuccinimidyl (sNHS) group. In other embodiments, the tissue binding moiety can comprise an antibody. In other embodiments, the tissue binding moiety can comprise a lipid which inserts into a cell membrane.

The first click motif and the second click motif are selected, as discussed below, such that the first click motif is capable of chemically reacting with the second click motif to form a covalent bond. In some examples, the first click motif can comprise a tetrazine (Tz) and the second click motif can comprise an alkene (e.g., a cyclooctene, such as trans-cyclooctene (TCO)). In other examples, the first click motif can comprise an azide and the second click motif comprises an alkyne (e.g., a cyclooctyne, such as dibenzocyclooctyne (DBCO)).

In some embodiments, L¹ is absent. In other embodiments, L¹ is present. In some embodiments, L² represents a cleavable linker (e.g., a hydrolysable linker, an enzymatically cleavable linker, a photocleavable linker, or a click cleavable linker).

In some embodiments, the active agent can comprise a diagnostic agent. In other embodiments, the active agent can comprise a therapeutic agent. In certain embodiments, the therapeutic agent can comprise an anti-cancer drug, a drug that promotes wound healing, a drug that promotes vascularization, a drug that treats or prevents infection, a drug that prevent restenosis, a drug that reduces macular degeneration, a drug that prevents immunological rejection, a drug that prevents thrombosis, or a drug that treats inflammation.

In some embodiments, contacting the target tissue with a Click Target comprises injecting or infusing a pharmaceutical composition comprising the Click Target into the target tissue. In some embodiments, contacting the target tissue with a Click Prodrug comprises systemically administering the Click Prodrug to the subject. Systemic administration can comprise, for example, administering the Click Prodrug to the subject orally, buccally, sublingually, rectally, intravenously, intra-arterially, intraosseously, intra-muscularly, intracerebrally, intracerebroventricularly, intrathecally, subcutaneously, intraperitoneally, intraocularly, intranasally, transdermally, epidurally, intracranially, percutaneously, intravaginaly, intrauterineally, intravitreally, transmucosally, or via injection, via aerosol-based delivery, or via implantation.

In some embodiments, the methods described herein can further comprise contacting the target tissue with one or more additional Click Prodrugs defined by Formula II

A-L²-CM² Formula II

wherein A represents an active agent; L¹ is absent, or represents a linking group; and CM² represents a second click motif complementary to the first click motif. This can include administering a second, third, fourth, fifth, sixth, seventh, eighth, ninth, tenth, etc. Click Prodrugs to the subject. In some cases, these subsequent doses can be administered of intervals of at least one day, at least one week, or at least one month. The active agent in each subsequent Click Prodrug can be the same or different than the active agent in the first Click Prodrug.

For example, in some cases, the active agent in each subsequent Click Prodrug can be the same as the active agent in the first Click Prodrug. In these embodiments, these subsequent doses can serve to “reload” the in situ depot with a further dose of the active agent. In other cases, the active agent in each subsequent Click Prodrug can be different than the active agent in the first Click Prodrug. In these embodiments, these subsequent doses can serve to deliver a active agent (e.g., for purposes of administering a combination therapy, or for purposes of altering the therapeutic strategy).

### DESCRIPTION OF DRAWINGS

FIGS. 1A and 1B schematically illustrate methods for drug delivery to a target tissue. As shown in FIG. 1A, a target tissue is first exposed to a “Click-Target,” decorating the tissue with a moiety that can participate in a click reaction. In this example, the “Click-Target” includes an amine-reactive NHS (N-hydroxysuccinimide) ester or sulfo-NHS ester and an azide moiety. The amine-reactive NHS (N-hydroxysuccinimide) ester or sulfo-NHS ester reacts with amines present in extracellular matrix (ECM) proteins to form a covalent bond. As shown in FIG. 1B, following treatment with the “Click-Target,” ECM proteins within the target tissue display azide moieties covalently tethered to the ECM proteins (1). The target tissue is then contacted with a “Click Prodrug.” In this example, the “Click Prodrug” includes an active agent (e.g., a therapeutic, diagnostic, or prophylactic agent) conjugated to a second moiety that can participate in a click reaction (a second “click motif”) through a cleavable bivalent linker. In this example, the second click motif is a diarylcyclooctyne moiety, which can participate in a click reaction with the azide moieties tethered to the ECM proteins to form a covalent bond. As a result, the active agent becomes covalently tethered to the ECM proteins within the target tissues (2). Subsequently, the cleavable bivalent linker in the “Click Prodrug” is cleaved via hydrolysis, releasing the active agent into the target tissue over time (3).

FIGS. 2A-2D illustrate the parameters and results of COMSOL modeling of the diffusion of an example “Click-Target” through a tumor.

FIGS. 3A-3C show the efficacy of intradermal administration of DBCO-cy7 (a model “Click Prodrug”).

FIGS. 4A-4E show the delivery of an example “Click Prodrug” (DBCO-cy7) to a tumor previously injected with an example “Click-Target” (azide-sNHS).

FIG. 5A shows the trendline made to predict the dose of “Click Prodrug” caught at the depot site.

FIG. 5B shows a histology image of a pancreatic tumor following delivery of a dose of “Click Prodrug.”

FIG. 6 shows a histology image from cryostat slices of tumors stained with DAPI and DBCO-cy3.

FIGS. 7A and 7B show an explanted tumor with no dbco-cy7 refill injection put through iDisco clearing, stained with DBCO-AF647, and imaged on a light sheet microscope.

FIG. 7A shows the tumor infused with a “Click-Target” and “Click Prodrug.” FIG. 7B shows a control.

FIG. 8 shows the delivery of an example “Click Prodrug” (Cy5-TCO) to tissued previously treated with an example “Click-Target” (methyl tetrazine-sNHS ester).

FIG. 9 is a plot showing that azide-sNHS ester depots allow long-term and repeated targeting with no apparent immunogenicity and are mutually compatible with tetrazine-TCO targeting for spatial separation of different regiments. FIG. 9 shows quantitation of systemic targeting of intradermal depots over the long term for 0.2M azide-sNHS (circles) or controlled PBS (squares) injections (50 μL).

FIG. 10 shows a timeline of depot refilling over 24 hours. Systemically administered fluorophore is initially present everywhere in the mouse, but after 24 hours is present specifically at injected site. Mice received intradermal injection of azide-sNHS (50 μL of 0.2M) or PBS were administered i.v. DBCO-Cy7. Mice were IVIS imaged before the dose and after 5 mins, 1 hour, and 24 hours.

FIG. 11 shows the click-specific capture of small molecules in the brain. Representative images of azide-sNHS and control mice after 24 hours after i.v. administration of DBCO-Cy7. The brain was removed and imaged alone.

FIG. 12 is a plot illustrating the click-specific capture of small molecules in the brain. Quantitation of intracranial ROI radiant efficiency was measured 24 hours after i.v. DBCO-Cy7 administration. Samples show mean+/−SEM. *=p<0.05, **=p<0.01 by Student's T-test.

FIG. 13 illustrates that azide-labeling of glioblastoma enables targeting. Mice bearing U87 GFP-expressing glioblastoma (green) were administered azide-sNHS (2 uL) intratumorally. AF647-DBCO (100 uL, 50 mg/1, red) was given i.v. Brains were submitted to iDisco clearing and imaged by light-sheet microscopy. Whole brain shown by background autofluorescence.

FIG. 14 shows the imaging NHS-ester depot distribution within a mouse brain using model fluorescent NHS ester and tissue clearing. Green isosurfaces of three Alexa Fluor 488-NHS ester injected tumors are shown as well as PBS-injected controls. Autofluorescence in tissue was visualized and set as a gray background.

FIG. 15 is a plot imaging NHS-ester distribution within a murine tumor using a fluorescent NHS ester and histology. Pancreatic tumors were injected with AF647-NHS. Tumors were removed, sectioned into 10 um section and stained with Picro Sirius Red to label collagen. Histological sections were imaged for picro Sirius red (left) or AF647 (middle).

FIG. 16 demonstrates that azide-sNHS ester depots allow long-term and repeated targeting with no apparent immunogenicity. H&E staining of skin injection site at one month for CD1 mice injected intradermally with 50 μL of azide-sNHS showing no difference between the two groups at any of the organs tested.

FIG. 17 demonstrates that azide-sNHS ester depots are mutually compatible with tetrazine-sNHS ester depots for spatial separation of different regiments. 50 μL of methyltetrazine sNHS (right, 0.05M) or azide-sNHS (left, 0.05M) was injected intradermally on the dorsal flank of 4 mice. 200 μL of 1:1 DBCO-Cy7/TCO-Cy5 were injected i.v. and imaged on the IVIS after 24 hours under the Cy7 and Cy5 filters.

FIGS. 18A-18B show the click-specific capture of checkpoint blockade PD-1 antibodies at pancreatic tumor sites. FIG. 18A show extracted azide-sNHS infused tumors (top row) and PBS (bottom row) 24 hours after i.v. dosing with DBCO- and Cy7-conjugated anti-PD1 antibody. FIG. 18B show the quantification of radiant efficiency over tumor and underlying carcass ROI's 24 hours after i.v. DBCO-Cy7-antibody administration. Samples show mean+/−SEM. *=p<0.05 by Student's T-test.

### DETAILED DESCRIPTION

### Definitions

In order that the present invention may be more readily understood, certain term are first defined.

Unless otherwise defined herein, scientific and technical terms used in connection with the present invention shall have the meanings that are commonly understood by those of ordinary skill in the art. The meaning and scope of the terms should be clear, however, in the event of any latent ambiguity, definitions provided herein take precedent over any dictionary or extrinsic definition.

The use of the terms “a” and “an” and “the” and similar referents in the context of describing the invention (especially in the context of the following claims) are to be construed to cover both the singular and the plural (i.e., one or more), unless otherwise indicated herein or clearly contradicted by context. The terms “comprising, “having,” “including,” and “containing” are to be construed as open-ended terms (i.e., meaning “including, but not limited to”) unless otherwise noted. Recitation of ranges of values herein are merely intended to serve as a shorthand method of referring individually to each separate value recited or falling within the range, unless otherwise indicated herein, and each separate value is incorporated into the specification as if it were individually recited. Ranges provided herein are understood to be shorthand for all of the values within the range. For example, a range of 1 to 50 is understood to include any number, combination of numbers, from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or sub-ranges from the group consisting of 10-40, 20-50, 5-35, etc.

The term “n-membered” where n is an integer typically describes the number of ring-forming atoms in a moiety where the number of ring-forming atoms is n. For example, piperidinyl is an example of a 6-membered heterocycloalkyl ring, pyrazolyl is an example of a 5-membered heteroaryl ring, pyridyl is an example of a 6-membered heteroaryl ring, and 1,2,3,4-tetrahydro-naphthalene is an example of a 10-membered cycloalkyl group.

As used herein, the phrase “optionally substituted” means unsubstituted or substituted. As used herein, the term “substituted” means that a hydrogen atom is removed and replaced by a substituent. It is to be understood that substitution at a given atom is limited by valency.

Throughout the definitions, the term “Cₙ₋ₘ” indicates a range which includes the endpoints, wherein n and m are integers and indicate the number of carbons. Examples include C₁₋₄, C_{1_6}, and the like.

As used herein, the term “Cₙ₋ₘ alkyl”, employed alone or in combination with other terms, refers to a saturated hydrocarbon group that may be straight-chain or branched, having n to m carbons. Examples of alkyl moieties include, but are not limited to, chemical groups such as methyl, ethyl, n-propyl, isopropyl, n-butyl, tert-butyl, isobutyl, sec-butyl; higher homologs such as 2-methyl-1-butyl, n-pentyl, 3-pentyl, n-hexyl, 1,2,2-trimethylpropyl, and the like. In some embodiments, the alkyl group contains from 1 to 6 carbon atoms, from 1 to 4 carbon atoms, from 1 to 3 carbon atoms, or 1 to 2 carbon atoms.

As used herein, “Cₙ₋ₘ alkenyl” refers to an alkyl group having one or more double carbon-carbon bonds and having n to m carbons. Example alkenyl groups include, but are not limited to, ethenyl, n-propenyl, isopropenyl, n-butenyl, sec-butenyl, and the like. In some embodiments, the alkenyl moiety contains 2 to 6, 2 to 4, or 2 to 3 carbon atoms.

As used herein, “Cₙ₋ₘ alkynyl” refers to an alkyl group having one or more triple carbon-carbon bonds and having n to m carbons. Example alkynyl groups include, but are not limited to, ethynyl, propyn-1-yl, propyn-2-yl, and the like. In some embodiments, the alkynyl moiety contains 2 to 6, 2 to 4, or 2 to 3 carbon atoms.

As used herein, the term “Cₙ₋ₘ alkylene”, employed alone or in combination with other terms, refers to a divalent alkyl linking group having n to m carbons. Examples of alkylene groups include, but are not limited to, ethan-1,2-diyl, propan-1,3-diyl, propan-1,2-diyl, butan-1,4-diyl, butan-1,3-diyl, butan-1,2-diyl, 2-methyl-propan-1,3-diyl, and the like. In some embodiments, the alkylene moiety contains 2 to 6, 2 to 4, 2 to 3, 1 to 6, 1 to 4, or 1 to 2 carbon atoms.

As used herein, the term “Cₙ₋ₘ alkoxy”, employed alone or in combination with other terms, refers to a group of formula —O-alkyl, wherein the alkyl group has n to m carbons. Example alkoxy groups include methoxy, ethoxy, propoxy (e.g., n-propoxy and isopropoxy), tert-butoxy, and the like. In some embodiments, the alkyl group has 1 to 6, 1 to 4, or 1 to 3 carbon atoms.

As used herein, the term “Cₙ₋ₘ alkylamino” refers to a group of formula —NH(alkyl), wherein the alkyl group has n to m carbon atoms. In some embodiments, the alkyl group has 1 to 6, 1 to 4, or 1 to 3 carbon atoms.

As used herein, the term “Cₙ₋ₘ alkoxycarbonyl” refers to a group of formula —C(O)-alkyl, wherein the alkyl group has n to m carbon atoms. In some embodiments, the alkyl group has 1 to 6, 1 to 4, or 1 to 3 carbon atoms.

As used herein, the term “Cₙ₋ₘ alkylcarbonyl” refers to a group of formula —C(O)-alkyl, wherein the alkyl group has n to m carbon atoms. In some embodiments, the alkyl group has 1 to 6, 1 to 4, or 1 to 3 carbon atoms.

As used herein, the term “Cₙ₋ₘ alkylcarbonylamino” refers to a group of formula —NHC(O)-alkyl, wherein the alkyl group has n to m carbon atoms. In some embodiments, the alkyl group has 1 to 6, 1 to 4, or 1 to 3 carbon atoms.

As used herein, the term “Cₙ₋ₘ alkylsulfonylamino” refers to a group of formula —NHS(O)₂-alkyl, wherein the alkyl group has n to m carbon atoms. In some embodiments, the alkyl group has 1 to 6, 1 to 4, or 1 to 3 carbon atoms.

As used herein, the term “aminosulfonyl” refers to a group of formula —S(O)₂NH₂.

As used herein, the term “Cₙ₋ₘ alkylaminosulfonyl” refers to a group of formula —S(O)₂NH(alkyl), wherein the alkyl group has n to m carbon atoms. In some embodiments, the alkyl group has 1 to 6, 1 to 4, or 1 to 3 carbon atoms.

As used herein, the term “di(Cₙ₋ₘ alkyl)aminosulfonyl” refers to a group of formula —S(O)₂N(alkyl)₂, wherein each alkyl group independently has n to m carbon atoms. In some embodiments, each alkyl group has, independently, 1 to 6, 1 to 4, or 1 to 3 carbon atoms.

As used herein, the term “aminosulfonylamino” refers to a group of formula —NHS(O)₂NH₂.

As used herein, the term “Cₙ₋ₘ alkylaminosulfonylamino” refers to a group of formula —NHS(O)₂NH(alkyl), wherein the alkyl group has n to m carbon atoms. In some embodiments, the alkyl group has 1 to 6, 1 to 4, or 1 to 3 carbon atoms.

As used herein, the term “di(Cₙ₋ₘ alkyl)aminosulfonylamino” refers to a group of formula —NHS(O)₂N(alkyl)₂, wherein each alkyl group independently has n to m carbon atoms. In some embodiments, each alkyl group has, independently, 1 to 6, 1 to 4, or 1 to 3 carbon atoms.

As used herein, the term “aminocarbonylamino”, employed alone or in combination with other terms, refers to a group of formula —NHC(O)NH₂.

As used herein, the term “Cₙ₋ₘ alkylaminocarbonylamino” refers to a group of formula —NHC(O)NH(alkyl), wherein the alkyl group has n to m carbon atoms. In some embodiments, the alkyl group has 1 to 6, 1 to 4, or 1 to 3 carbon atoms.

As used herein, the term “di(Cₙ₋ₘ alkyl)aminocarbonylamino” refers to a group of formula —NHC(O)N(alkyl)₂, wherein each alkyl group independently has n to m carbon atoms. In some embodiments, each alkyl group has, independently, 1 to 6, 1 to 4, or 1 to 3 carbon atoms.

As used herein, the term “Cₙ₋ₘ alkylcarbamyl” refers to a group of formula —C(O)—NH(alkyl), wherein the alkyl group has n to m carbon atoms. In some embodiments, the alkyl group has 1 to 6, 1 to 4, or 1 to 3 carbon atoms.

As used herein, the term “thio” refers to a group of formula —SH.

As used herein, the term “Cₙ₋ₘ alkylsulfinyl” refers to a group of formula —S(O)-alkyl, wherein the alkyl group has n to m carbon atoms. In some embodiments, the alkyl group has 1 to 6, 1 to 4, or 1 to 3 carbon atoms.

As used herein, the term “Cₙ₋ₘ alkylsulfonyl” refers to a group of formula —S(O)₂-alkyl, wherein the alkyl group has n to m carbon atoms. In some embodiments, the alkyl group has 1 to 6, 1 to 4, or 1 to 3 carbon atoms.

As used herein, the term “amino” refers to a group of formula —NH₂.

As used herein, the term “aryl,” employed alone or in combination with other terms, refers to an aromatic hydrocarbon group, which may be monocyclic or polycyclic (e.g., having 2, 3 or 4 fused rings). The term “Cₙ₋ₘ aryl” refers to an aryl group having from n to m ring carbon atoms. Aryl groups include, e.g., phenyl, naphthyl, anthracenyl, phenanthrenyl, indanyl, indenyl, and the like. In some embodiments, aryl groups have from 6 to about 20 carbon atoms, from 6 to about 15 carbon atoms, or from 6 to about 10 carbon atoms. In some embodiments, the aryl group is a substituted or unsubstituted phenyl.

As used herein, the term “carbamyl” to a group of formula —C(O)NH₂.

As used herein, the term “carbonyl”, employed alone or in combination with other terms, refers to a —C(═O)— group, which may also be written as C(O).

As used herein, the term “di(Cₙ₋ₘ-alkyl)amino” refers to a group of formula —N(alkyl)₂, wherein the two alkyl groups each has, independently, n to m carbon atoms. In some embodiments, each alkyl group independently has 1 to 6, 1 to 4, or 1 to 3 carbon atoms.

As used herein, the term “di(Cₙ₋ₘ-alkyl)carbamyl” refers to a group of formula —C(O)N(alkyl)₂, wherein the two alkyl groups each has, independently, n to m carbon atoms. In some embodiments, each alkyl group independently has 1 to 6, 1 to 4, or 1 to 3 carbon atoms.

As used herein, the term “halo” refers to F, Cl, Br, or I. In some embodiments, a halo is F, Cl, or Br. In some embodiments, a halo is F or Cl.

As used herein, “Cₙ₋ₘ haloalkoxy” refers to a group of formula —O-haloalkyl having n to m carbon atoms. An example haloalkoxy group is OCF₃. In some embodiments, the haloalkoxy group is fluorinated only. In some embodiments, the alkyl group has 1 to 6, 1 to 4, or 1 to 3 carbon atoms.

As used herein, the term “Cₙ₋ₘ haloalkyl”, employed alone or in combination with other terms, refers to an alkyl group having from one halogen atom to 2s+1 halogen atoms which may be the same or different, where “s” is the number of carbon atoms in the alkyl group, wherein the alkyl group has n to m carbon atoms. In some embodiments, the haloalkyl group is fluorinated only. In some embodiments, the alkyl group has 1 to 6, 1 to 4, or 1 to 3 carbon atoms.

As used herein, “cycloalkyl” refers to non-aromatic cyclic hydrocarbons including cyclized alkyl and/or alkenyl groups. Cycloalkyl groups can include mono- or polycyclic (e.g., having 2, 3 or 4 fused rings) groups and spirocycles. Cycloalkyl groups can have 3, 4, 5, 6, 7, 8, 9, or 10 ring-forming carbons (C₃₋₁₀). Ring-forming carbon atoms of a cycloalkyl group can be optionally substituted by oxo or sulfido (e.g., C(O) or C(S)). Cycloalkyl groups also include cycloalkylidenes. Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, and the like. In some embodiments, cycloalkyl is cyclopropyl, cyclobutyl, cyclopentyl, cyclohexyl, cyclopentyl, or adamantyl. In some embodiments, the cycloalkyl has 6-10 ring-forming carbon atoms. In some embodiments, cycloalkyl is adamantyl. Also included in the definition of cycloalkyl are moieties that have one or more aromatic rings fused (i.e., having a bond in common with) to the cycloalkyl ring, for example, benzo or thienyl derivatives of cyclopentane, cyclohexane, and the like. A cycloalkyl group containing a fused aromatic ring can be attached through any ring-forming atom including a ring-forming atom of the fused aromatic ring.

As used herein, “heteroaryl” refers to a monocyclic or polycyclic aromatic heterocycle having at least one heteroatom ring member selected from sulfur, oxygen, and nitrogen. In some embodiments, the heteroaryl ring has 1, 2, 3, or 4 heteroatom ring members independently selected from nitrogen, sulfur and oxygen. In some embodiments, any ring-forming N in a heteroaryl moiety can be an N-oxide. In some embodiments, the heteroaryl has 5-10 ring atoms and 1, 2, 3 or 4 heteroatom ring members independently selected from nitrogen, sulfur and oxygen. In some embodiments, the heteroaryl has 5-6 ring atoms and 1 or 2 heteroatom ring members independently selected from nitrogen, sulfur and oxygen. In some embodiments, the heteroaryl is a five-membered or six-membereted heteroaryl ring. A five-membered heteroaryl ring is a heteroaryl with a ring having five ring atoms wherein one or more (e.g., 1, 2, or 3) ring atoms are independently selected from N, O, and S. Exemplary five-membered ring heteroaryls are thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4-oxadiazolyl. A six-membered heteroaryl ring is a heteroaryl with a ring having six ring atoms wherein one or more (e.g., 1, 2, or 3) ring atoms are independently selected from N, O, and S. Exemplary six-membered ring heteroaryls are pyridyl, pyrazinyl, pyrimidinyl, triazinyl and pyridazinyl.

As used herein, “heterocycloalkyl” refers to non-aromatic monocyclic or polycyclic heterocycles having one or more ring-forming heteroatoms selected from O, N, or S. Included in heterocycloalkyl are monocyclic 4-, 5-, 6-, and 7-membered heterocycloalkyl groups. Heterocycloalkyl groups can also include spirocycles. Example heterocycloalkyl groups include pyrrolidin-2-one, 1,3-isoxazolidin-2-one, pyranyl, tetrahydropuran, oxetanyl, azetidinyl, morpholino, thiomorpholino, piperazinyl, tetrahydrofuranyl, tetrahydrothienyl, piperidinyl, pyrrolidinyl, isoxazolidinyl, isothiazolidinyl, pyrazolidinyl, oxazolidinyl, thiazolidinyl, imidazolidinyl, azepanyl, benzazapene, and the like. Ring-forming carbon atoms and heteroatoms of a heterocycloalkyl group can be optionally substituted by oxo or sulfido (e.g., C(O), S(O), C(S), or S(O)₂, etc.). The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds. Also included in the definition of heterocycloalkyl are moieties that have one or more aromatic rings fused (i.e., having a bond in common with) to the cycloalkyl ring, for example, benzo or thienyl derivatives of piperidine, morpholine, azepine, etc. A heterocycloalkyl group containing a fused aromatic ring can be attached through any ring-forming atom including a ring-forming atom of the fused aromatic ring. In some embodiments, the heterocycloalkyl has 4-10, 4-7 or 4-6 ring atoms with 1 or 2 heteroatoms independently selected from nitrogen, oxygen, or sulfur and having one or more oxidized ring members.

At certain places, the definitions or embodiments refer to specific rings (e.g., an azetidine ring, a pyridine ring, etc.). Unless otherwise indicated, these rings can be attached to any ring member provided that the valency of the atom is not exceeded. For example, an azetidine ring may be attached at any position of the ring, whereas a pyridin-3-yl ring is attached at the 3-position.

The term “direct bond” or “bond” refers to a single, double or triple bond between two groups. In certain embodiments, a “direct bond” refers to a single bond between two groups

The term “compound” as used herein is meant to include all stereoisomers, geometric isomers, tautomers, and isotopes of the structures depicted. Compounds herein identified by name or structure as one particular tautomeric form are intended to include other tautomeric forms unless otherwise specified.

Compounds provided herein also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, enamine-imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

In some embodiments, the compounds described herein can contain one or more asymmetric centers and thus occur as racemates and racemic mixtures, enantiomerically enriched mixtures, single enantiomers, individual diastereomers and diastereomeric mixtures (e.g., including (R)- and (S)-enantiomers, diastereomers, (D)-isomers, (L)-isomers, (+) (dextrorotatory) forms, (−) (levorotatory) forms, the racemic mixtures thereof, and other mixtures thereof). Additional asymmetric carbon atoms can be present in a substituent, such as an alkyl group. All such isomeric forms, as well as mixtures thereof, of these compounds are expressly included in the present description. The compounds described herein can also or further contain linkages wherein bond rotation is restricted about that particular linkage, e.g. restriction resulting from the presence of a ring or double bond (e.g., carbon-carbon bonds, carbon-nitrogen bonds such as amide bonds). Accordingly, all cis/trans and E/Z isomers and rotational isomers are expressly included in the present description. Unless otherwise mentioned or indicated, the chemical designation of a compound encompasses the mixture of all possible stereochemically isomeric forms of that compound.

Optical isomers can be obtained in pure form by standard procedures known to those skilled in the art, and include, but are not limited to, diastereomeric salt formation, kinetic resolution, and asymmetric synthesis. See, for example, Jacques, et al., Enantiomers, Racemates and Resolutions (Wiley Interscience, New York, 1981); Wilen, S. H., et al., Tetrahedron 33:2725 (1977); Eliel, E. L. Stereochemistry of Carbon Compounds (McGraw-Hill, N Y, 1962); Wilen, S. H. Tables of Resolving Agents and Optical Resolutions p. 268 (E. L. Eliel, Ed., Univ. of Notre Dame Press, Notre Dame, Ind. 1972), each of which is incorporated herein by reference in their entireties. It is also understood that the compounds described herein include all possible regioisomers, and mixtures thereof, which can be obtained in pure form by standard separation procedures known to those skilled in the art, and include, but are not limited to, column chromatography, thin-layer chromatography, and high-performance liquid chromatography.

Unless specifically defined, compounds provided herein can also include all isotopes of atoms occurring in the intermediates or final compounds. Isotopes include those atoms having the same atomic number but different mass numbers. Unless otherwise stated, when an atom is designated as an isotope or radioisotope (e.g., deuterium, [¹¹C], [¹⁸F]), the atom is understood to comprise the isotope or radioisotope in an amount at least greater than the natural abundance of the isotope or radioisotope. For example, when an atom is designated as “D” or “deuterium”, the position is understood to have deuterium at an abundance that is at least 3000 times greater than the natural abundance of deuterium, which is 0.015% (i.e., at least 45% incorporation of deuterium).

All compounds, and pharmaceutically acceptable salts thereof, can be found together with other substances such as water and solvents (e.g. hydrates and solvates) or can be isolated.

In some embodiments, preparation of compounds can involve the addition of acids or bases to affect, for example, catalysis of a desired reaction or formation of salt forms such as acid addition salts.

Example acids can be inorganic or organic acids and include, but are not limited to, strong and weak acids. Some example acids include hydrochloric acid, hydrobromic acid, sulfuric acid, phosphoric acid, p-toluenesulfonic acid, 4-nitrobenzoic acid, methanesulfonic acid, benzenesulfonic acid, trifluoroacetic acid, and nitric acid. Some weak acids include, but are not limited to acetic acid, propionic acid, butanoic acid, benzoic acid, tartaric acid, pentanoic acid, hexanoic acid, heptanoic acid, octanoic acid, nonanoic acid, and decanoic acid.

Example bases include lithium hydroxide, sodium hydroxide, potassium hydroxide, lithium carbonate, sodium carbonate, potassium carbonate, and sodium bicarbonate. Some example strong bases include, but are not limited to, hydroxide, alkoxides, metal amides, metal hydrides, metal dialkylamides and arylamines, wherein; alkoxides include lithium, sodium and potassium salts of methyl, ethyl and t-butyl oxides; metal amides include sodium amide, potassium amide and lithium amide; metal hydrides include sodium hydride, potassium hydride and lithium hydride; and metal dialkylamides include lithium, sodium, and potassium salts of methyl, ethyl, n-propyl, iso-propyl, n-butyl, tert-butyl, trimethylsilyl and cyclohexyl substituted amides.

In some embodiments, the compounds provided herein, or salts thereof, are substantially isolated. By “substantially isolated” is meant that the compound is at least partially or substantially separated from the environment in which it was formed or detected. Partial separation can include, for example, a composition enriched in the compounds provided herein. Substantial separation can include compositions containing at least about 50%, at least about 60%, at least about 70%, at least about 80%, at least about 90%, at least about 95%, at least about 97%, or at least about 99% by weight of the compounds provided herein, or salt thereof. Methods for isolating compounds and their salts are routine in the art.

The expressions, “ambient temperature” and “room temperature” or “rt” as used herein, are understood in the art, and refer generally to a temperature, e.g. a reaction temperature, that is about the temperature of the room in which the reaction is carried out, for example, a temperature from about 20° C. to about 30° C.

As used herein, the term “bioorthogonal” or “bioorthogonal functional group” refer to a functional group or chemical reaction that can occur inside a living cell, tissue, or organism without interfering with native biological or biochemical processes. A bioorthogonal functional group or reaction is not toxic to cells. In some embodiments, the first click motif present on the Click Target comprises a bioorthogonal functional group and the second click motif present on the Click Prodrug comprises a complementary functional group capable of chemically reacting with the first click motif to form a covalent bond.

Compositions, Systems, and Methods

Disclosed herein are compositions, systems, and methods for the delivery (e.g., local and sustained delivery) of an active agent to a target tissue. The strategies employ click chemistry to deliver a drug to a tissue of interest (where it is later released). Target tissue could be, for example, a tumor, an area of ischemia (heart attack, stroke), an area of local infection, an area of immunological organ injection, or other localized disease.

First, the tissue of interest is functionalized by injecting a reactive “click” molecule (referred to as a “Click-Target”) into the target tissue. The “Click-Target” is a bifunctional molecule including a first moiety that interacts (e.g., forms a covalent bond with) the target tissue, and a second moiety that can participate in a click reaction (a first “click motif”). By way of example, the first moiety can react to form a covalent bond with the proteins in the target tissue. These proteins could be part of the extracellular matrix or could be proteins on cells. The result is a tissue that is decorated with the “Click-Target” molecule, and thus display the second moiety that can participate in a click reaction (the first “click motif”). Optionally, the “Click-Target” con further include a linker (e.g., a bivalent linker) covalently joining the first moiety and the second moiety. When present, the linker may be a cleavable (e.g., hydrolysable) linker or a non-cleavable linker (if desired for a particular active agent and/or

“Click Prodrug,” as discussed below).

Bioorthogonal “click” chemistry is then used to target drugs specifically to the “Click-Target.” Click chemistry refers to a class chemical reaction between two click groups that exhibit good yields, wide functional group tolerance, and are highly selective even in the presence of a complex mixture of biological molecules. These characteristics allow the click reactions to proceed even in vivo.

In a second step, a drug conjugate (referred to as a “Click Prodrug”) is administered to the subject. The “Click Prodrug” can include an active agent (e.g., a therapeutic, diagnostic, or prophylactic agent) conjugated to a second moiety that can participate in a click reaction (a second “click motif”) directly, or optionally through a linker (e.g., a bivalent linker). When present, the linker may be a cleavable (e.g., hydrolysable) linker or a non-cleavable linker (if desired for a particular active agent and/or “Click-Target”). The first “click motif” and the second “click motif” are selected such that they can react through a click reaction to form a covalent bond. Generally, the “Click Prodrugs” do not cross cell membranes and do not have activity (or toxicity) on their own. This can reduce systemic toxicity associated with administration of the active agent. However, the “Click Prodrug” does have the ability to “click” at the location of the “Click-Target,” thereby anchoring the active agent at the target tissue (e.g., the site of disease) that has been pre-labeled with the “Click Target.”

If desired for a particular application, the “Click Prodrug” can include an active agent (e.g., a therapeutic agent) conjugated to a second moiety that can participate in a click reaction (a second “click motif”) through a cleavable (e.g., hydrolysable) linker. In these cases, once the “Click Prodrug” has been bound to the “Click-Target,” the cleavable linker tethering the active agent to the “Click-Target” can be cleaved (e.g., via hydrolysis), releasing the active agent into the target tissue. Based on the nature of the cleavable linker, the cleavage rate (and by extension the drug delivery profile of the active agent) can be tuned to provide controlled deliver of the active agent.

In other cases, the “Click Prodrug” can include an active agent conjugated to a second moiety that can participate in a click reaction (a second “click motif”) directly or through a non-cleavable linker. In these cases, once the “Click Prodrug” has been bound to the “Click-Target,” the active agent remains bound to the “Click Target,” assuming the “Click-Target” does not include a cleavable linker. This can allow for an active agent (e.g., a therapeutic agent such as a radionuclide, or a diagnostic agent such as an imaging agent) to be retained (i.e., not released) into the target tissue.

Accordingly, provided herein are methods for delivering an active agent to a target tissue. These methods can comprise (i) contacting the target tissue with a Click Target defined by Formula I

X-L¹-CM¹ Formula I

wherein X represents a tissue binding moiety; L¹ is absent, or represents a linking group; and CM¹ represents a first click motif; and (ii) contacting the target tissue with a Click Prodrug defined by Formula II

A-L²-CM² Formula II

wherein A represents an active agent; L¹ is absent, or represents a linking group; and CM² represents a second click motif complementary to the first click motif. The identity of the first click motif and the second click motif are selected, as discussed below, such that the first click motif is capable of chemically reacting with the second click motif to form a covalent bond.

In some embodiments, contacting the target tissue with a Click Target comprises injecting or infusing a pharmaceutical composition comprising the Click Target into the target tissue. In some embodiments, contacting the target tissue with a Click Prodrug comprises systemically administering the Click Prodrug to the subject. Systemic administration can comprise, for example, administering the Click Prodrug to the subject orally, buccally, sublingually, rectally, intravenously, intra-arterially, intraosseously, intra-muscularly, intracerebrally, intracerebroventricularly, intrathecally, subcutaneously, intraperitoneally, intraocularly, intranasally, transdermally, epidurally, intracranially, percutaneously, intravaginaly, intrauterineally, intravitreally, transmucosally, or via injection, via aerosol-based delivery, or via implantation. In other embodiments, the step of contacting the target tissue with a Click Target can comprise topically administering the Click Target to a tumor and/or tissue surrounding a tumor during or after a surgical procedure to resect a tumor.

In some embodiments, the methods described herein can further comprise contacting the target tissue with one or more additional Click Prodrugs defined by Formula II

A-L²-CM² Formula II

wherein A represents an active agent; L¹ is absent, or represents a linking group; and CM² represents a second click motif complementary to the first click motif. This can include administering a second, third, fourth, fifth, sixth, seventh, eighth, ninth, tenth, etc. Click Prodrugs to the subject. In some cases, these subsequent doses can be administered of intervals of at least one day, at least one week, or at least one month. The active agent in each subsequent Click Prodrug can be the same or different than the active agent in the first Click Prodrug.

For example, in some cases, the active agent in each subsequent Click Prodrug can be the same as the active agent in the first Click Prodrug. In these embodiments, these subsequent doses can serve to “reload” the in situ depot with a further dose of the active agent. In other cases, the active agent in each subsequent Click Prodrug can be different than the active agent in the first Click Prodrug. In these embodiments, these subsequent doses can serve to deliver a active agent (e.g., for purposes of administering a combination therapy, or for purposes of altering the therapeutic strategy).

The target tissue can comprise any tissue in a subject which might benefit (e.g., therapeutically, prophylactically, or diagnostically) from the local delivery of an active agent. In certain embodiments, the target tissue can comprise a solid tumor.

In certain cases, the tissue can comprise tissue associated with a local cancer (e.g., pancreatic cancer, glioblastoma, breast cancer, or hepacellular carcinoma), or tissue associated with a peritoneal cancer (e.g., a sarcoma, ovarian cancer, or mesothelioma). In certain cases, the tissue can comprise a tissue associated with a local infection (e.g., an implant-associated infection, osteomyelitis). In certain cases, the tissue can comprise a transplanted tissue (e.g., an organ transplant). In certain cases, the tissue can comprise a blood contacting surface (e.g., a segment of vasculature (e.g., to prevent restenosis or thrombosis, for example, following implantation of a stent). In certain cases, the tissue can comprise a wound (e.g., to improve wound healing and regeneration).

Also provided are methods of maintaining or reducing the size of a tumor in a subject in need thereof. These methods can comprise injecting or infusing into the tumor a Click Target defined by Formula I

X-L¹-CM¹ Formula I

wherein X represents a tissue binding moiety; L¹ is absent, or represents a linking group; and CM¹ represents a first click motif; and administering to the subject a Click Prodrug defined by Formula II

A-L²-CM² Formula II

wherein A represents an anti-cancer drug; L¹ is absent, or represents a linking group; and CM² represents a second click motif complementary to the first click motif; and optionally repeating step (ii) one or more times, thereby maintaining or reducing the size of the tumor in the subject.

Also provided are methods of treating a tumor in a subject that comprise surgically resecting the tumor or a portion thereof; contacting tissue surrounding the resected tumor with a Click Target defined by Formula I

X-L¹-CM¹ Formula I

wherein X represents a tissue binding moiety; L¹ is absent, or represents a linking group; and CM¹ represents a first click motif; administering to the subject a Click Prodrug defined by Formula II

A-L²-CM² Formula II

wherein A represents an anti-cancer drug; L¹ is absent, or represents a linking group; and CM² represents a second click motif complementary to the first click motif; and (iii) optionally repeating step (ii) one or more times, thereby maintaining or reducing the size of the tumor in the subject.

Also provided are kits for administering an active agent to a subject in need thereof comprising a first pharmaceutical composition comprising a Click Target defined by Formula I and a pharmaceutically acceptable carrier

X-L¹-CM¹ Formula I

wherein X represents a tissue binding moiety; L¹ is absent, or represents a linking group; and CM¹ represents a first click motif; and a second pharmaceutical composition comprising a Click Prodrug defined by Formula II and a pharmaceutically acceptable carrier

A-L²-CM² Formula II

wherein A represents an active agent; L¹ is absent, or represents a linking group; and CM² represents a second click motif complementary to the first click motif.

The first click motif and the second click motif are selected, as discussed below, such that the first click motif is capable of chemically reacting with the second click motif to form a covalent bond. In some examples, the first click motif can comprise a tetrazine (Tz) and the second click motif can comprise an alkene (e.g., a cyclooctene, such as trans-cyclooctene (TCO)). In other examples, the first click motif can comprise an azide and the second click motif comprises an alkyne (e.g., a cyclooctyne, such as dibenzocyclooctyne (DBCO)).

Tissue Binding Moieties

The Click Target can include any suitable tissue binding moiety. The tissue binding moiety can be any moiety which functions to anchor the Click Target in the target tissue. For example, in some embodiments, the tissue binding moiety can comprise a functional group capable of chemically reacting with a functional group in a peptide (e.g., an amine group, a thiol group, a carboxylate group, or a phenol group) to form a covalent bond.

In certain embodiments, the tissue binding moiety comprises a functional group capable of chemically reacting with an amine group in a peptide (e.g., an extracellular matrix protein) to form a covalent bond, such as a hydroxysuccinimidyl (NHS) group or a sulfo-hydroxysuccinimidyl (sNHS) group. Other groups that activate carboxylic acids and react with amines, including acyl chlorides, isocyanate groups, sulfonyl chloride groups, aldehyde groups, acyl azide groups, anhydrides, fluorobenzene groups, carbonates, imidoester groups, epoxides and fluorophenyl esters, can also be used. In other embodiments, the tissue binding moiety comprises a functional group capable of chemically reacting with a thiol group in a peptide to form a covalent bond, such as a maleimide group or an iodoacetate group. Other suitable functional groups are described, for example, in Montalbetti, C.A.G.N. and Falque, V. “Amide bond formation and peptide coupling,” Tetrahedron, 2015, 61: 10827-10852, which is hereby incorporated by reference in its entirety.

In other embodiments, the tissue binding moiety can be a moiety with associates non-covalently with the target tissue. For example, the tissue binding moiety can be an antibody that binds to the target tissue. In another example, the tissue binding moiety can comprise a lipid which inserts into a cell membrane.

Linking Groups

When present, the linking group can be any suitable group or moiety which is at minimum bivalent, and connects the two radical moieties to which the linking group is attached in the compounds described herein. The linking group can be composed of any assembly of atoms, including oligomeric and polymeric chains. In some cases, the total number of atoms in the linking group can be from 3 to 200 atoms (e.g., from 3 to 150 atoms, from 3 to 100 atoms, from 3 and 50 atoms, from 3 to 25 atoms, from 3 to 15 atoms, or from 3 to 10 atoms).

In some embodiments, the linking group can be, for example, an alkyl, alkoxy, alkylaryl, alkylheteroaryl, alkylcycloalkyl, alkylheterocycloalkyl, alkylthio, alkylsulfinyl, alkylsulfonyl, alkylamino, dialkylamino, alkylcarbonyl, alkoxycarbonyl, alkylaminocarbonyl, dialkylaminocarbonyl, or polyamino group. In some embodiments, the linking group can comprises one of the groups above joined to one or both of the moieties to which it is attached by a functional group. Examples of suitable functional groups include, for example, secondary amides (—CONH—), tertiary amides (—CONR—), secondary carbamates (—OCONH—; —NHCOO—), tertiary carbamates (—OCONR—; —NRCOO—), ureas (—NHCONH—; —NRCONH—; —NHCONR—, or —NRCONR—), carbinols (—CHOH—, —CROH—), ethers (—O—), and esters (—COO—, —CH₂O₂C—, CHRO₂C—), wherein R is an alkyl group, an aryl group, or a heterocyclic group. For example, in some embodiments, the linking group can comprise an alkyl group (e.g., a C₁-C₁₂ alkyl group, a C₁-C₈ alkyl group, or a C₁-C₆ alkyl group) bound to one or both of the moieties to which it is attached via an ester (—COO—, —CH₂O₂C—, CHRO₂C—), a secondary amide (—CONH—), or a tertiary amide (—CONR—), wherein R is an alkyl group, an aryl group, or a heterocyclic group. In certain embodiments, the linking group can be chosen from one of the following:

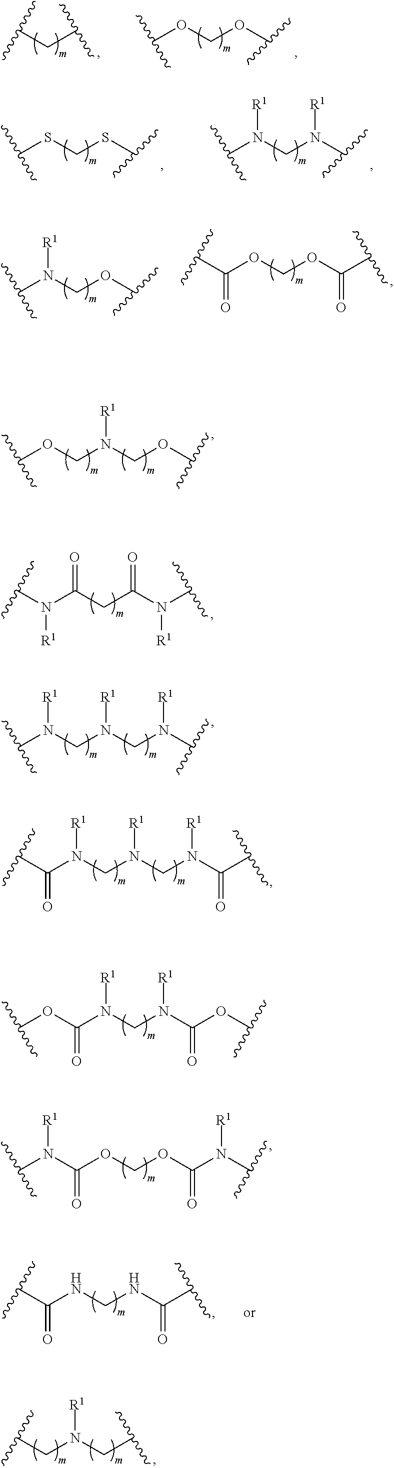

where m is an integer from 1 to 12 and R¹ is, independently for each occurrence, hydrogen, an alkyl group, an aryl group, or a heterocyclic group.

If desired, the linker can serve to modify the solubility of the compounds described herein. In some embodiments, the linker is hydrophilic. In some embodiments, the linker can be an alkyl group, an alkylaryl group, an oligo- or polyalkylene oxide chain (e.g., an oligo- or polyethylene glycol chain), or an oligo- or poly(amino acid) chain.

In certain embodiments, the linker can be cleavable (e.g., cleavable by hydrolysis under physiological conditions, enzymatically cleavable, or a combination thereof). Examples of cleavable linkers include a hydrolysable linker, a pH cleavage linker, an enzyme cleavable linker, or disulfide bonds that are cleaved through reduction by free thiols and other reducing agents; peptide bonds that are cleaved through the action of proteases and peptidase; nucleic acid bonds cleaved through the action of nucleases; esters that are cleaved through hydrolysis either by enzymes or through the action of water in vivo; hydrazones, acetals, ketals, oximes, imine, aminals and similar groups that are cleaved through hydrolysis in the body; photo-cleavable bonds that are cleaved by the exposure to a specific wavelength of light; mechano-sensitive groups that are cleaved through the application of ultrasound or a mechanical strain (e.g., a mechanical strain created by a magnetic field on a magneto-responsive gel). In other embodiments, the linker can be “click cleavable” (i.e., a click-to-release linker). Such linkers are cleaved when a click motif to which the linker is bound participates in a click reaction. Examples of click cleavable linkers (and associated click motifs) are known in the art. See, for example, Versteegen et al. Angew. Chem. Int. Ed., 2018, 57(33): 10494-10499; Versteegen et al. Angew. Chem. Int. Ed., 2013, 52(52): 14112-14116; U.S. Patent Application Publication No. 2019/0247513; and U.S. Pat. No. 10,004,810; each of which is hereby incorporated by reference in its entirety. In embodiments where an external stimulus (e.g., irradiation by light or application of a magnetic field) induces cleavage, the methods described herein can further comprise the step of applying the external stimulus to induce cleavage.

In other embodiments, the linker can be non-cleavable. In some cases, non-cleavable linker(s) can be utilized with it is desirable that the active agent be retained (as opposed to released from) the Click Target. This can be the case, for example, when the active agent is an imaging agent (e.g., a contrast agent), an agent for photodynamic therapy, or a radionuclide.

Click Motifs

Example click motif pairs used as the first click motif and the second click motif include, but not limited to, azide with phosphine; azide with cyclooctyne; nitrone with cyclooctyne; nitrile oxide with norbornene; oxanorbornadiene with azide; trans-cyclooctene with s-tetrazine; quadricyclane with bis(dithiobenzil)nickel(II).

In some embodiments, the second click motif comprises an alkene, e.g., a cyclooctene, e.g., a transcyclooctene (TCO) or norbornene (NOR), and the first click motif comprises a tetrazine (Tz). In other embodiments, the second click motif comprises an alkyne, e.g., a cyclooctyne such as dibenzocyclooctyne (DBCO), and the first click motif comprises an azide (Az). In some embodiments, the second click motif comprises a Tz, and the first click motif comprises an alkene such as transcyclooctene (TCO) or norbornene (NOR). Alternatively or in addition, the first click motif comprises an Az, and the second click motif comprises a cyclooctyne such as dibenzocyclooctyne (DBCO). TCO reacts specifically in a click chemistry reaction with a tetrazine (Tz) moiety. DBCO reacts specifically in a click chemistry reaction with an azide (Az) moiety. Norbornene reacts specifically in a click chemistry reaction with a tetrazine (Tz) moiety.

Exemplary click chemistry reactions (and by extension click motifs) are shown below. For example, copper(I)-catalyzed Azide-Alkyne Cycloaddition (CuAAC) comprises using a Copper (Cu) catalyst at room temperature. The Azide-Alkyne Cycloaddition is a 1,3-dipolar cycloaddition between an azide and a terminal or internal alkyne to give a 1,2,3-triazole.

Another example of click chemistry includes Staudinger ligation, which is a reaction that is based on the classic Staudinger reaction of azides with triarylphosphines. It launched the field of bioorthogonal chemistry as the first reaction with completely abiotic functional. The azide acts as a soft electrophile that prefers soft nucleophiles such as phosphines. This is in contrast to most biological nucleophiles which are typically hard nucleophiles. The reaction proceeds selectively under water-tolerant conditions to produce a stable product. Phosphines are completely absent from living systems and do not reduce disulfide bonds despite mild reduction potential. Azides had been shown to be biocompatible in FDA-approved drugs such as azidothymidine and through other uses as cross linkers. Additionally, their small size allows them to be easily incorporated into biomolecules through cellular metabolic pathways

Copper-free click chemistry is a bioorthogonal reaction first developed by Carolyn Bertozzi as an activated variant of an azide alkyne cycloaddition. Unlike CuAAC, Cu-free click chemistry has been modified to be bioorthogonal by eliminating a cytotoxic copper catalyst, allowing reaction to proceed quickly and without live cell toxicity. Instead of copper, the reaction is a strain-promoted alkyne-azide cycloaddition (SPAAC). It was developed as a faster alternative to the Staudinger ligation, with the first generations reacting over sixty times faster. The incredible bioorthogonality of the reaction has allowed the Cu-free click reaction to be applied within cultured cells, live zebrafish, and mice. Cyclooctynes were selected as the smallest stable alkyne ring which increases reactivity through ring strain which has calculated to be 19.9 kcal/mol.

Copper-free click chemistry also includes nitrone dipole cycloaddition. Copper-free click chemistry has been adapted to use nitrones as the 1,3-dipole rather than azides and has been used in the modification of peptides.

This cycloaddition between a nitrone and a cyclooctyne forms N-alkylated isoxazolines. The reaction rate is enhanced by water and is extremely fast with second order rate constants ranging from 12 to 32 M⁻¹·s⁻¹, depending on the substitution of the nitrone. Although the reaction is extremely fast, incorporating the nitrone into biomolecules through metabolic labeling has only been achieved through post-translational peptide modification.

Another example of click chemistry includes norbornene cycloaddition. 1,3 dipolar cycloadditions have been developed as a bioorthogonal reaction using a nitrile oxide as a 1,3-dipole and a norbornene as a dipolarophile. Its primary use has been in labeling DNA and RNA in automated oligonucleotide synthesizers.

Norbornenes were selected as dipolarophiles due to their balance between strain-promoted reactivity and stability. The drawbacks of this reaction include the cross-reactivity of the nitrile oxide due to strong electrophilicity and slow reaction kinetics.

Another example of click chemistry includes oxanorbornadiene cycloaddition. The oxanorbornadiene cycloaddition is a 1,3-dipolar cycloaddition followed by a retro-Diels Alder reaction to generate a triazole-linked conjugate with the elimination of a furan molecule. This reaction is useful in peptide labeling experiments, and it has also been used in the generation of SPECT imaging compounds.

Ring strain and electron deficiency in the oxanorbornadiene increase reactivity towards the cycloaddition rate-limiting step. The retro-Diels Alder reaction occurs quickly afterwards to form the stable 1,2,3 triazole. Limitations of this reaction include poor tolerance for substituents which may change electronics of the oxanorbornadiene and low rates (second order rate constants on the order of 10⁻⁴).

Another example of click chemistry includes tetrazine ligation. The tetrazine ligation is the reaction of a trans-cyclooctene and an s-tetrazine in an inverse-demand Diels Alder reaction followed by a retro-Diels Alder reaction to eliminate nitrogen gas. The reaction is extremely rapid with a second order rate constant of 2000 M⁻¹-s⁻¹ (in 9:1 methanol/water) allowing modifications of biomolecules at extremely low concentrations.

The highly strained trans-cyclooctene is used as a reactive dienophile. The diene is a 3,6-diaryl-s-tetrazine which has been substituted in order to resist immediate reaction with water. The reaction proceeds through an initial cycloaddition followed by a reverse Diels Alder to eliminate N₂ and prevent reversibility of the reaction.

Not only is the reaction tolerant of water, but it has been found that the rate increases in aqueous media. Reactions have also been performed using norbornenes as dienophiles at second order rates on the order of 1 M⁻¹·s⁻¹ in aqueous media. The reaction has been applied in labeling live cells and polymer coupling.

Another example of click chemistry includes is [4+1] cycloaddition. This isocyanide click reaction is a [4+1] cycloaddition followed by a retro-Diels Alder elimination of N₂.

The reaction proceeds with an initial [4+1] cycloaddition followed by a reversion to eliminate a thermodynamic sink and prevent reversibility. This product is stable if a tertiary amine or isocyanopropanoate is used. If a secondary or primary isocyanide is used, the produce will form an imine which is quickly hydrolyzed.

Isocyanide is a favored chemical reporter due to its small size, stability, non-toxicity, and absence in mammalian systems. However, the reaction is slow, with second order rate constants on the order of 10⁻²M⁻¹·s⁻¹.

Another example of click chemistry includes quadricyclane ligation. The quadricyclane ligation utilizes a highly strained quadricyclane to undergo [2+2+2] cycloaddition with π systems.

Quadricyclane is abiotic, unreactive with biomolecules (due to complete saturation), relatively small, and highly strained (^{˜}80 kcal/mol). However, it is highly stable at room temperature and in aqueous conditions at physiological pH. It is selectively able to react with electron-poor π systems but not simple alkenes, alkynes, or cyclooctynes.

Bis(dithiobenzil)nickel(II) was chosen as a reaction partner out of a candidate screen based on reactivity. To prevent light-induced reversion to norbornadiene, diethyldithiocarbamate is added to chelate the nickel in the product.

These reactions are enhanced by aqueous conditions with a second order rate constant of 0.25 M⁻¹·s⁻¹. Of particular interest is that it has been proven to be bioorthogonal to both oxime formation and copper-free click chemistry.

The exemplary click chemistry reactions have high specificity, efficient kinetics, and occur in vivo under physiological conditions. See, e.g., Baskin et al. Proc. Natl. Acad. Sci. USA 104(2007):16793; Oneto et al. Acta biomaterilia (2014); Neves et al. Bioconjugate chemistry 24(2013):934; Koo et al. Angewandte Chemie 51(2012):11836; and Rossin et al. Angewandte Chemie 49(2010):3375. For a review of a wide variety of click chemistry reactions and their methodologies, see e.g., Nwe K and Brechbiel M W, 2009 Cancer Biotherapy and Radiopharmaceuticals, 24(3): 289-302; Kolb H C et al., 2001 Angew. Chem. Int. Ed. 40: 2004-2021. The entire contents of each of the foregoing references are incorporated herein by reference.

Exemplary click motif pairs are shown in the table below.

| Functional | | | |
|---|---|---|---|
| group/Click | Paired | | Reaction type |
| Motif | with | Functional group/Click Motif | (Reference) |
| Azide | | Phosphine | Staudinger ligation |
| | | | (Saxon et al. Science |
| | | | 287(2000):2007-10) |
| azide | | Cyclooctyne, e.g., dibenzocyclooctyne, | Copper-free click |
| | | one of the cyclooctynes shown below, or | chemistry (Jewett et |
| | | other similar cyclooctynes: | al. J. Am. Chem. Soc. |
| | | 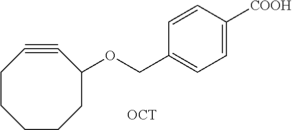 | 132.11(2010):3688- 90; Sletten et al. Organic Letters 10.14 (2008)3097-9; Lutz. Angew. Chem., Int. Ed 47.12(2008):2182) |
| | | 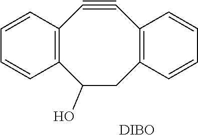 | |
| | | 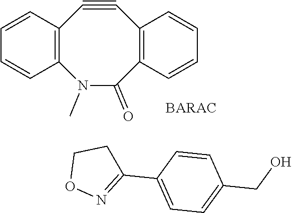 | |
| | | 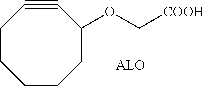 [SMILES: O=C(O)COC1C#CCCCCC1] | |
| | | 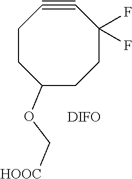 | |
| | | 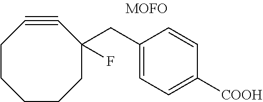 [SMILES: O=C(O)c1ccc(CC2(F)C#CCCCCC2)cc1] | |
| | | 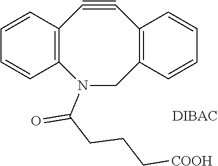 [SMILES: O=C(O)CCCC(=O)N1Cc2ccccc2C#Cc2ccccc21] | |
| | | 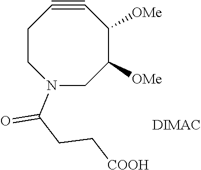 | |
| Nitrone | | Cyclooctyne | Nitrone Dipole |
| | | | Cycloaddition (Ning |
| | | | et al. Angew. Chem., |
| | | | Int. Ed 49.17 |
| | | | (2010):3065) |
| Nitrile oxide | | Norbornene | Norbornene |
| | | | Cycloaddition |
| | | | (Gutsmiedl et al. |
| | | | Organic Letters |
| | | | 11.11(2009):2405-8) |
| Oxanorbornad- | | Azide | Oxanorbornadiene |
| iene | | | Cycloaddition (Van |
| | | | Berkel et al. |
| | | | ChemBioChem |
| | | | 8.13(2007):1504-8) |
| Trans- | | s-tetrazine | Tetrazine ligation |
| cyclooctene, | | | (Hansell et al. J. Am. |
| norbornene, | | | Chem. Soc. |
| or other | | | 133.35(2011):13828- |
| alkene | | | 31) |
| Nitrile | | 1,2,4,5-tetrazine | [4 + 1] cycloaddition |
| | | | (Stackman et al. |
| | | | Organic and Biomol. |
| | | | Chem. |
| | | | 9.21(2011):7303) |
| Quadricyclane | | Bis(dithiobenzil)nickel(II) | Quadricyclane |
| | | | Ligation (Sletten et |
| | | | al. J. Am. Chem. Soc. |
| | | | 133.44(2011):17570- |
| | | | 3) |
| Ketone or | | Hydrazines, hydrazones, oximes, amines, | Non-aldol carbonyl |
| aldehyde | | ureas, thioureas, etc. | chemistry |
| | | | (Khomyakova EA, et |
| | | | al. |
| | | | Nucleosides |
| | | | Nucleotides Nucleic |
| | | | Acids. 30(7-8) |
| | | | (2011) 577-84 |
| Thiol | | Maleimide | Michael addition |
| | | | (Zhou et al. |
| | | | Bioconjug Chem |
| | | | 2007 18(2):323-32.) |
| Dienes | | Dienophiles | Diels Alder (Rossin |
| | | | et al. Nucl Med. |
| | | | (2013) 54(11):1989- |
| | | | 95) |
| Tetrazine | | norbornene, propene, trans-cyclooctene, | |
| | | other strained alkenes. | |

Other suitable include the motifs can be found, for example, in Patterson, D. M., et al. “Finding the Right (Bioorthogonal) Chemistry,” ACS Chem. Biol., 2014, 9(3): 592-605; Akgun, B., et al. “Synergic “Click” Boronate/Thiosemicarbazone System for Fast and Irreversible Bioorthogonal Conjugation in Live Cells,” J. Am. Chem. Soc., 2017, 139(40): 14285-14291; and Akgun, B. and Hall, D. G. “Fast and Tight Boronate Formation for Click Bioorthogonal Conjugation,” Angew. Chem., Int. Ed. 2016, 55(12): 3909-3913, each of which is hereby incorporated by reference in its entirety.

Active Agents

The term “Active Agent”, as used herein, refers to a physiologically or pharmacologically active substance that acts locally and/or systemically in the body. An active agent is a substance that is administered to a patient for the treatment (e.g., therapeutic agent), prevention (e.g., prophylactic agent), or diagnosis (e.g., diagnostic agent) of a disease or disorder.

The active agent can be a small molecule, or a biologic. A biologic is a medicinal product manufactured in, extracted from, or semi-synthesized from biological sources which is different from chemically synthesized pharmaceuticals. In some embodiments, biologics used as the active agent can include, for example, antibodies, blood components, allergenics, gene therapies, and recombinant therapeutic proteins. Biologics can comprise, for example, sugars, proteins, or nucleic acids, and they can be isolated from natural sources such as human, animal, or microorganism.

In some embodiments, the active agent can comprise an anti-cancer drug, a drug that promotes wound healing, a drug that treats or prevents infection, or a drug that promotes vascularization. For example, the active agent can comprise an anti-cancer drug, such as a chemotherapeutic or a cancer vaccine. The anti-cancer drug can include a small molecule, a peptide or polypeptide, a protein or fragment thereof (e.g., an antibody or fragment thereof), or a nucleic acid.

Exemplary anti-cancer drugs can include, but are not limited to, Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Adrucil (Fluorouracil), Afatinib Dimaleate, Afinitor (Everolimus), Aldara (Imiquimod), Aldesleukin, Alemtuzumab, Alimta (Pemetrexed Disodium), Aloxi (Palonosetron Hydrochloride), Ambochlorin (Chlorambucil), Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Avastin (Bevacizumab), Axitinib, Azacitidine, BEACOPP, Bendamustine Hydrochloride, BEP, Bevacizumab, Bexarotene, Bexxar (Tositumomab and I 131 Iodine Tositumomab), Bicalutamide, Bleomycin, Bortezomib, Bosulif (Bosutinib), Bosutinib, Brentuximab Vedotin, Busulfan, Busulfex (Busulfan), Cabazitaxel, Cabozantinib-S-Malate, CAF, Campath (Alemtuzumab), Camptosar (Irinotecan Hydrochloride), Capecitabine, CAPDX, Carboplatin, Carboplatin-Taxol, Carfilzomib, Casodex (Bicalutamide), CeeNU (Lomustine), Cerubidine (Daunorubicin Hydrochloride), Cervarix (Recombinant HPV Bivalent Vaccine), Cetuximab, Chlorambucil, Chlorambucil-Prednisone, CHOP, Cisplatin, Clafen (Cyclophosphamide), Clofarabine, Clofarex (Clofarabine), Clolar (Clofarabine), CMF, Cometriq (Cabozantinib-S-Malate), COPP, COPP-ABV, Cosmegen (Dactinomycin), Crizotinib, CVP, Cyclophosphamide, Cyfos (Ifosfamide), Cytarabine, Cytarabine (Liposomal), Cytosar-U (Cytarabine), Cytoxan (Cyclophosphamide), Dabrafenib, Dacarbazine, Dacogen (Decitabine), Dactinomycin, Dasatinib, Daunorubicin Hydrochloride, Decitabine, Degarelix, Denileukin Diftitox, Denosumab, DepoCyt (Liposomal Cytarabine), DepoFoam (Liposomal Cytarabine), Dexrazoxane Hydrochloride, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Efudex (Fluorouracil), Elitek (Rasburicase), Ellence (Epirubicin Hydrochloride), Eloxatin (Oxaliplatin), Eltrombopag Olamine, Emend (Aprepitant), Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Etopophos (Etoposide Phosphate), Etoposide, Etoposide Phosphate, Evacet (Doxorubicin Hydrochloride Liposome), Everolimus, Evista (Raloxifene Hydrochloride), Exemestane, Fareston (Toremifene), Faslodex (Fulvestrant), FEC, Femara (Letrozole), Filgrastim, Fludara (Fludarabine Phosphate), Fludarabine Phosphate, Fluoroplex (Fluorouracil), Fluorouracil, Folex (Methotrexate), Folex PFS (Methotrexate), Folfiri, Folfiri-Bevacizumab, Folfiri-Cetuximab, Folfirinox, Folfox, Folotyn (Pralatrexate), FU-LV, Fulvestrant, Gardasil (Recombinant HPV Quadrivalent Vaccine), Gazyva (Obinutuzumab), Gefitinib, Gemcitabine Hydrochloride, Gemcitabine-Cisplatin, Gemcitabine-Oxaliplatin, Gemtuzumab Ozogamicin, Gemzar (Gemcitabine Hydrochloride), Gilotrif (Afatinib Dimaleate), Gleevec (Imatinib Mesylate), Glucarpidase, Goserelin Acetate, Halaven (Eribulin Mesylate), Herceptin (Trastuzumab), HPV Bivalent Vaccine (Recombinant), HPV Quadrivalent Vaccine (Recombinant), Hycamtin (Topotecan Hydrochloride), Hyper-CVAD, Ibritumomab Tiuxetan, Ibrutinib, ICE, Iclusig (Ponatinib Hydrochloride), Ifex (Ifosfamide), Ifosfamide, Ifosfamidum (Ifosfamide), Imatinib Mesylate, Imbruvica (Ibrutinib), Imiquimod, Inlyta (Axitinib), Intron A (Recombinant Interferon Alfa-2b), Iodine 131 Tositumomab and Tositumomab, Ipilimumab, Iressa (Gefitinib), Irinotecan Hydrochloride, Istodax (Romidepsin), Ixabepilone, Ixempra (Ixabepilone), Jakafi (Ruxolitinib Phosphate), Jevtana (Cabazitaxel), Kadcyla (Ado-Trastuzumab Emtansine), Keoxifene (Raloxifene Hydrochloride), Kepivance (Palifermin), Kyprolis (Carfilzomib), Lapatinib Ditosylate, Lenalidomide, Letrozole, Leucovorin Calcium, Leukeran (Chlorambucil), Leuprolide Acetate, Levulan (Aminolevulinic Acid), Linfolizin (Chlorambucil), LipoDox (Doxorubicin Hydrochloride Liposome), Liposomal Cytarabine, Lomustine, Lupron (Leuprolide Acetate), Lupron Depot (Leuprolide Acetate), Lupron Depot-Ped (Leuprolide Acetate), Lupron Depot-3 Month (Leuprolide Acetate), Lupron Depot-4 Month (Leuprolide Acetate), Marqibo (Vincristine Sulfate Liposome), Matulane (Procarbazine Hydrochloride), Mechlorethamine Hydrochloride, Megace (Megestrol Acetate), Megestrol Acetate, Mekinist (Trametinib), Mercaptopurine, Mesna, Mesnex (Mesna), Methazolastone (Temozolomide), Methotrexate, Methotrexate LPF (Methotrexate), Mexate (Methotrexate), Mexate-AQ (Methotrexate), Mitomycin C, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Nelarabine, Neosar (Cyclophosphamide), Neupogen (Filgrastim), Nexavar (Sorafenib Tosylate), Nilotinib, Nolvadex (Tamoxifen Citrate), Nplate (Romiplostim), Obinutuzumab, Ofatumumab, Omacetaxine Mepesuccinate, Oncaspar (Pegaspargase), Ontak (Denileukin Diftitox), OEPA, OPPA, Oxaliplatin, Paclitaxel, Paclitaxel Albumin-stabilized Nanoparticle Formulation, Palifermin, Palonosetron Hydrochloride, Pamidronate Disodium, Panitumumab, Paraplat (Carboplatin), Paraplatin (Carboplatin), Pazopanib Hydrochloride, Pegaspargase, Peginterferon Alfa-2b, PEG-Intron (Peginterferon Alfa-2b), Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Pralatrexate, Prednisone, Procarbazine Hydrochloride, Proleukin (Aldesleukin), Prolia (Denosumab), Promacta (Eltrombopag Olamine), Provenge (Sipuleucel-T), Purinethol (Mercaptopurine), Radium 223 Dichloride, Raloxifene Hydrochloride, Rasburicase, R-CHOP, R-CVP, Recombinant HPV Bivalent Vaccine, Recombinant HPV Quadrivalent Vaccine, Recombinant Interferon Alfa-2b, Regorafenib, Revlimid (Lenalidomide), Rheumatrex (Methotrexate), Rituxan (Rituximab), Rituximab, Romidepsin, Romiplostim, Rubidomycin (Daunorubicin Hydrochloride), Ruxolitinib Phosphate, Sclerosol Intrapleural Aerosol (Talc), Sipuleucel-T, Sorafenib Tosylate, Sprycel (Dasatinib), Stanford V, Sterile Talc Powder (Talc), Steritalc (Talc), Stivarga (Regorafenib), Sunitinib Malate, Sutent (Sunitinib Malate), Sylatron (Peginterferon Alfa-2b), Synovir (Thalidomide), Synribo (Omacetaxine Mepesuccinate), Tafinlar (Dabrafenib), Talc, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Temodar (Temozolomide), Temozolomide, Temsirolimus, Thalidomide, Thalomid (Thalidomide), Toposar (Etoposide), Topotecan Hydrochloride, Toremifene, Torisel (Temsirolimus), Tositumomab and I¹³¹ Iodine Tositumomab, Totect (Dexrazoxane Hydrochloride), Trametinib, Trastuzumab, Treanda (Bendamustine Hydrochloride), Trisenox (Arsenic Trioxide), Tykerb (Lapatinib Ditosylate), Vandetanib, VAMP, Vectibix (Panitumumab), VeIP, Velban (Vinblastine Sulfate), Velcade (Bortezomib), Velsar (Vinblastine Sulfate), Vemurafenib, VePesid (Etoposide), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, Vismodegib, Voraxaze (Glucarpidase), Vorinostat, Votrient (Pazopanib Hydrochloride), Wellcovorin (Leucovorin Calcium), Xalkori (Crizotinib), Xeloda (Capecitabine), XELOX, Xgeva (Denosumab), Xofigo (Radium 223 Dichloride), Xtandi (Enzalutamide), Yervoy (Ipilimumab), Zaltrap (Ziv-Aflibercept), Zelboraf (Vemurafenib), Zevalin (Ibritumomab Tiuxetan), Zinecard (Dexrazoxane Hydrochloride), Ziv-Aflibercept, Zoladex (Goserelin Acetate), Zoledronic Acid, Zolinza (Vorinostat), Zometa (Zoledronic Acid), and Zytiga (Abiraterone Acetate).

In some embodiments, the active agent can comprise a drug that promotes wound healing or vascularization. In some embodiments, the active agent can comprise a drug that reduces ischemia, e.g., due to peripheral artery disease (PAD) or damaged myocardial tissues due to myocardial infarction. For example, the drug can comprise a protein or fragment thereof, e.g., a growth factor or angiogenic factor, such as vascular endothelial growth factor (VEGF), e.g., VEGFA, VEGFB, VEGFC, or VEGFD, and/or IGF, e.g., IGF-1, fibroblast growth factor (FGF), angiopoietin (ANG) (e.g., Ang1 or Ang2), matrix metalloproteinase (MMP), delta-like ligand 4 (DLL4), or combinations thereof. Drugs that promote wound healing or vascularization are non-limiting, as the skilled artisan would be able to readily identify other drugs that promote wound healing or vascularization.

In some embodiments, the active agent can comprise an anti-proliferative drug, e.g., mycophenolate mofetil (MMF), azathioprine, sirolimus, tacrolimus, paclitaxel, biolimus A9, novolimus, myolimus, zotarolimus, everolimus, or tranilast. These anti-proliferative drugs are non-limiting, as the skilled artisan would be able to readily identify other anti-proliferative drugs.

In some embodiments, the active agent can comprise an anti-inflammatory drug, e.g., corticosteroid anti-inflammatory drugs (e.g., beclomethasone, beclometasone, budesonide, flunisolide, fluticasone propionate, triamcinolone, methylprednisolone, prednisolone, or prednisone); or non-steroidal anti-inflammatory drugs (NSAIDs) (e.g., acetylsalicylic acid, diflunisal, salsalate, choline magnesium trisalicylate, ibuprofen, dexibuprofen, naproxen, fenoprofen, ketoprofen, dexketoprofen, fluribiprofen, oxaprozin, loxoprofen, indomethacin, tolmetin, sulindac, etodolac, ketorolac, diclofenac, aceclofenac, nabumetone, piroxicam, meloxicam, tenoxicam, droxicam, lornoxicam, isoxicam, mefenamic acid, meclofenamic acid, flufenamic acid, tolfenamic acid, celecoxib, rofecoxib, valdecoxib, parecoxib, lumiracoxib, etoricoxib, firocoxib, nimesulide, licofelone, H-harpaide, or lysine clonixinate). These anti-inflammatory drugs are non-limiting, as the skilled artisan would be able to readily identify other anti-inflammatory drugs.

In some embodiments, the active agent can comprise a drug that prevents or reduces transplant rejection, e.g., an immunosuppressant. Exemplary immunosuppressants include calcineurin inhibitors (e.g., cyclosporine, Tacrolimus (FK506)); mammalian target of rapamycin (mTOR) inhibitors (e.g., rapamycin, also known as Sirolimus); antiproliferative agents (e.g., azathioprine, mycophenolate mofetil, mycophenolate sodium); antibodies (e.g., basiliximab, daclizumab, muromonab); corticosteroids (e.g., prednisone). These drugs that prevent or reduce transplant rejection are non-limiting, as the skilled artisan would be able to readily identify other drugs that prevent or reduce transplant rejection.

In some embodiments, the active agent can comprise an anti-thrombotic drug, e.g., an anti-platelet drug, an anticoagulant drug, or a thrombolytic drug.

Exemplary anti-platelet drugs include an irreversible cyclooxygenase inhibitor (e.g., aspirin or triflusal); an adenosine diphosphate (ADP) receptor inhibitor (e.g., ticlopidine, clopidogrel, prasugrel, or tricagrelor); a phosphodiesterase inhibitor (e.g., cilostazol); a glycoprotein IIB/IIIA inhibitor (e.g., abciximab, eptifibatide, or tirofiban); an adenosine reuptake inhibitor (e.g., dipyridamole); or a thromboxane inhibitor (e.g., thromboxane synthase inhibitor, a thromboxane receptor inhibitor, such as terutroban). These anti-platelet drugs are non-limiting, as the skilled artisan would be able to readily identify other anti-platelet drugs.

Exemplary anticoagulant drugs include coumarins (e.g., warfarin, acenocoumarol, phenprocoumon, atromentin, brodifacoum, or phenindione); heparin and derivatives thereof (e.g., heparin, low molecular weight heparin, fondaparinux, or idraparinux); factor Xa inhibitors (e.g., rivaroxaban, apixaban, edoxaban, betrixaban, darexaban, letaxaban, or eribaxaban); thrombin inhibitors (e.g., hirudin, lepirudin, bivalirudin, argatroban, or dabigatran); antithrombin protein; batroxobin; hementin; and thrombomodulin. These anticoagulant drugs are non-limiting, as the skilled artisan would be able to readily identify other anticoagulant drugs.

Exemplary thrombolytic drugs include tissue plasminogen activator (t-PA) (e.g., alteplase, reteplase, or tenecteplase); anistreplase; streptokinase; or urokinase.

In other embodiments, the active agent can comprise a drug that prevents restenosis, e.g., an anti-proliferative drug, an anti-inflammatory drug, or an anti-thrombotic drug. Exemplary anti-proliferative drugs, anti-inflammatory drugs, and anti-thrombotic drugs are described herein.

In some embodiments, the active agent can comprise a drug that treats or prevents infection, e.g., an antibiotic. Suitable antibiotics include, but are not limited to, beta-lactam antibiotics (e.g., penicillins, cephalosporins, carbapenems), polymyxins, rifamycins, lipiarmycins, quinolones, sulfonamides, macrolides lincosamides, tetracyclines, aminoglycosides, cyclic lipopeptides (e.g., daptomycin), glycylcyclines (e.g., tigecycline), oxazonidinones (e.g., linezolid), and lipiarmycines (e.g., fidazomicin). For example, antibiotics include erythromycin, clindamycin, gentamycin, tetracycline, meclocycline, (sodium) sulfacetamide, benzoyl peroxide, and azelaic acid. Suitable penicillins include amoxicillin, ampicillin, bacampicillin, carbenicillin, cloxacillin, dicloxacillin, flucloxacillin, mezlocillin, nafcillin, oxacillin, penicillin g, penicillin v, piperacillin, pivampicillin, pivmecillinam, and ticarcillin. Exemplary cephalosporins include cefacetrile, cefadroxil, cephalexin, cefaloglycin, cefalonium, cefaloridine, cefalotin, cefapirin, cefatrizine, cefazaflur, cefazedone, cefazolin, cefradine, cefroxadine, ceftezole, cefaclor, cefamandole, cefmetazole, cefonicid, cefotetan, cefoxitin, cefprozil, cefuroxime, cefuzonam, cfcapene, cefdaloxime, cefdinir, cefditoren, cefetamet, cefixime, cefmenoxime, cefodizime, cefotaxime, cefpimizole, cefpodoxime, cefteram, ceftibuten, ceftiofur, ceftiolene, ceftizoxime, ceftriaxone, ceftazidime, cefclidine, cefepime, ceflurprenam, cefoselis, cefozopran, cefpirome, cequinome, ceftobiprole, ceftaroline, cefaclomezine, cefaloram, cefaparole, cefcanel, cefedrlor, cefempidone, cefetrizole, cefivitril, cefmatilen, cefmepidium, cefovecin, cefoxazole, cefrotil, cefsumide, cefuracetime, and ceftioxide. Monobactams include aztreonam. Suitable carbapenems include imipenem/cilastatin, doripenem, meropenem, and ertapenem. Exemplary macrolides include azithromycin, erythromycin, larithromycin, dirithromycin, roxithromycin, and telithromycin. Lincosamides include clindamycin and lincomycin. Exemplary streptogramins include pristinamycin and quinupristin/dalfopristin. Suitable aminoglycoside antibiotics include amikacin, gentamycin, kanamycin, neomycin, netilmicin, paromomycin, streptomycin, and tobramycin. Exemplary quinolones include flumequine, nalidixic acid, oxolinic acid, piromidic acid, pipemidic acid, rosoxacin, ciprofloxacin, enoxacin, lomefloxacin, nadifloxacin, norfloxacin, ofoxacin, pefloxacin, rufloxacin, balofloxacin, gatifloxacin, repafloxacin, levofloxacin, moxifloxacin, pazufloxacin, sparfloxacin, temafloxacin, tosufloxacin, besifloxacin, clinafoxacin, gemifloxacin, sitafloxacin, trovafloxacin, and prulifloxacin. Suitable sulfonamides include sulfamethizole, sulfamethoxazole, and trimethoprim-sulfamethoxazone. Exemplary tetracyclines include demeclocycline, doxycycline, minocycline, oxytetracycline, tetracycline, and tigecycline. Other antibiotics include chloramphenicol, metronidazole, tinidazole, nitrofurantoin, vancomycin, teicoplanin, telavancin, linezolid, cycloserine, rifampin, rifabutin, rifapentin, bacitracin, polymyxin B, viomycin, and capreomycin. The skilled artisan could readily identify other antibiotics useful in the devices and methods described herein.

In some embodiments, the active agent can comprise a drug that reduces macular degeneration. One common current treatment for macular degeneration involves the injection of anti-angiogenesis compounds intraocularly (Lucentis, Eylea). The repeated intraocular injections are sometimes poorly tolerated by patients, leading to low patient compliance. As described herein, the ability to noninvasively refill drug depots for macular degeneration significantly improves patient compliance and patient tolerance of disease, e.g., macular degeneration, treatment. Controlled, repeated release made possible by the methods described herein allows for fewer drug dosings and improved patient comfort.

In some embodiments, the active agent can comprise a drug that prevents immunological rejection. Prior to the invention described herein, to prevent immunological rejection of cells, tissues or whole organs, patients required lifelong therapy of systemic anti-rejection drugs that cause significant side effects and deplete the immune system, leaving patients at greater risk for infection and other complications. The ability to locally release anti-rejection drugs and to repeatedly load Click Prodrug allows for more local anti-rejection therapy with fewer systemic side effects, improved tolerability and better efficacy.

In some embodiments, the active agent can comprise a drug that prevents thrombosis. Some vascular devices such as vascular grafts and coated stents suffer from thrombosis, in which the body mounts a thrombin-mediated response to the devices. Anti-thrombotic drugs, released from these devices, allows for temporary inhibition of the thrombosis process, but the devices have limited drugs and cannot prevent thrombosis once the drug supply is exhausted. Since these devices are implanted for long periods of time (potentially for the entire lifetime of the patient), temporary thrombosis inhibition is not sufficient. The ability to repeatedly and locally administer anti-thrombotic drugs and release the drug significantly improves clinical outcomes and allows for long-term thrombosis inhibition.

In some embodiments, the active agent can comprise a drug that treats inflammation. Chronic inflammation is characterized by persistent inflammation due to non-degradable pathogens, viral infections, or autoimmune reactions and can last years and lead to tissue destruction, fibrosis, and necrosis. In some cases, inflammation is a local disease, but clinical interventions are almost always systemic. Anti-inflammatory drugs given systemically have significant side-effects including gastrointestinal problems, cardiotoxicity, high blood pressure and kidney damage, allergic reactions, and possibly increased risk of infection. The ability to repeatedly and locally release anti-inflammatory drugs such as NSAIDs and COX-2 inhibitors could reduce these side effects. These methods can provide the ability to deliver long term and local anti-inflammatory care while avoiding systemic side effects.

Other suitable active agents include, for example, immunotherapeutics/immunoadjuvants such as checkpoint inhibitors and STING agonists and agonists for toll-like receptors. Examples include STING ligands (e.g., natural cyclic dinucleotides, cAIMP dinucleotide, fluorine-containing cyclic dinulcoetides, phosphorothioate-containing cyclic dinucleotides, DMXAA); TLR2 ligands; TLR3 ligands (e.g., poly(I:C)); TLR4 ligands (e.g., lipopolysaccharides, monophosphoryl lipid A, CRX-527); TLR5 ligands; TLR7/8 ligands (e.g., gardiquimod, imiquimod, loxoribine, resiquimod, imidazoquinolines, adenine base analogs, benzoazepine analogs); TLR9 ligands (e.g., natural CpG ODNs, phosphorothioate CpG ODNs); TLR13 ligands (e.g., rRNA-derived ODNs); and NOD ligands (e.g., iE-DAP, meso-lanthionine tripeptide, D-gamma-Glu-mDAP, L-Ala-gamm-D-Glu-mDAP).

Example Click Prodrugs

By way of example, representative Click Prodrugs are shown below.

| | | Click | |
|---|---|---|---|
| Active Agent | Linker | Motif | Example |
| Doxorubicin | Hydrazine | DBC O | 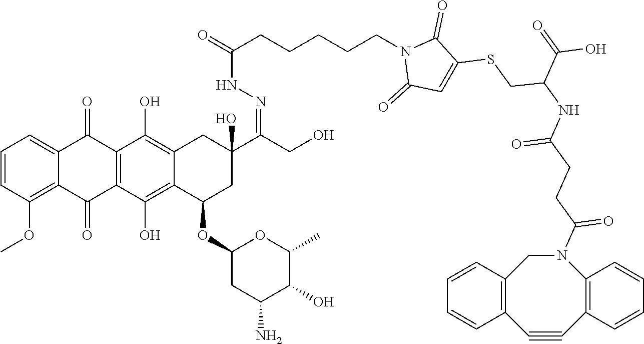 [SMILES: COc1cccc2c1C(=O)c1c(O)c3c(c(O)c1C2=O)C[C@](O)(/C(CO)=N\NC(=O)CCCCCN1C(=O)C=C(SCC(NC(=O)CCC(=O)N2Cc4ccccc4C#Cc4ccccc42)C(=O)O)C1=O)C[C@H]3O[C@@H]1C[C@@H](N)[C@@H](O)[C@@H](C)O1] |
| Paclitaxel | Ester | DBC O | 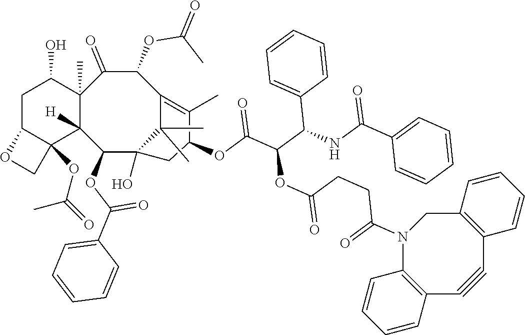 |
| Gemcitabine | Amide | DBC O | 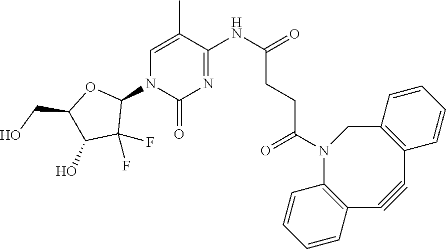 [SMILES: Cc1cn([C@@H]2O[C@H](CO)[C@@H](O)C2(F)F)c(=O)nc1NC(=O)CCC(=O)N1Cc2ccccc2C#Cc2ccccc21] |
| Topotecan | Ester | DBC O | 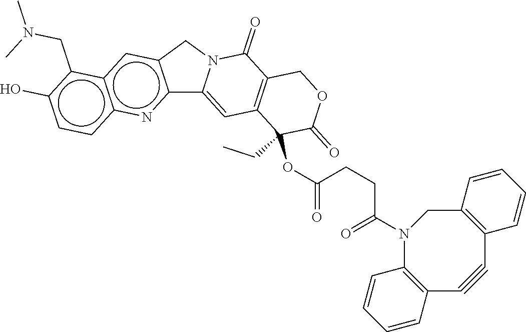 |
| Tacrolimus | Hydrazone | DBC O | 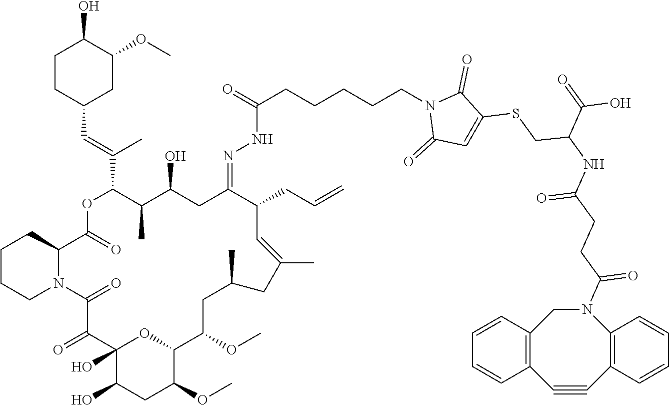 [SMILES: C=CC[C@@H]1/C=C(\C)C[C@H](C)C[C@H](OC)[C@H]2O[C@@](O)(C(=O)C(=O)N3CCCC[C@H]3C(=O)O[C@H](/C(C)=C/[C@@H]3CC[C@@H](O)[C@H](OC)C3)[C@H](C)[C@@H](O)C/C1=N/NC(=O)CCCCCN1C(=O)C=C(SCC(NC(=O)CCC(=O)N3Cc4ccccc4C#Cc4ccccc43)C(=O)O)C1=O)[C@H](O)C[C@@H]2OC] |
| Mycophenolic Acid | Ester | DBC O | 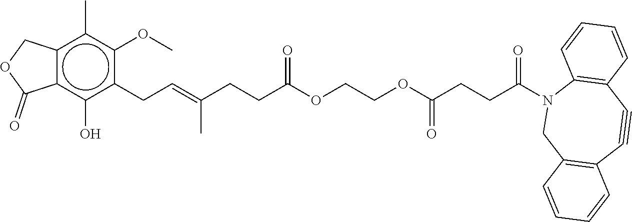 |
| Rapamycin | Hydrazone | DBC O | 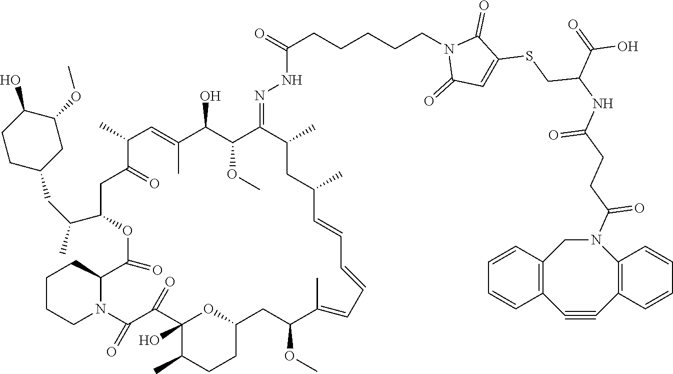 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)CC(=O)[C@H](C)/C=C(\C)[C@@H](O)[C@@H](OC)/C(=N/NC(=O)CCCCCN2C(=O)C=C(SCC(NC(=O)CCC(=O)N3Cc4ccccc4C#Cc4ccccc43)C(=O)O)C2=O)[C@H](C)C[C@H](C)/C=C/C=C/C=C/1C] |
| Resiquimod | Phenylsulfone | TCO | 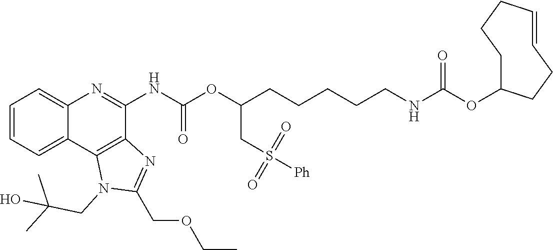 [SMILES: CCOCc1nc2c(NC(=O)OC(CCCCCNC(=O)OC3CC/C=C/CCC3)CS(=O)(=O)c3ccccc3)nc3ccccc3c2n1CC(C)(C)O] |
| Erlotinib | Phenylsulfone | TCO | 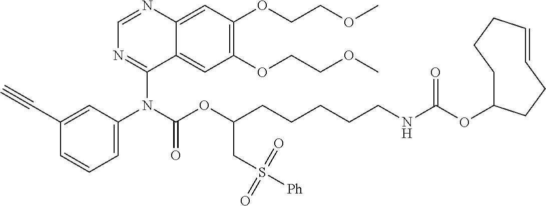 [SMILES: C#Cc1cccc(N(C(=O)OC(CCCCCNC(=O)OC2CC/C=C/CCC2)CS(=O)(=O)c2ccccc2)c2ncnc3cc(OCCOC)c(OCCOC)cc23)c1] |
| DMXAA | Ester | TCO | 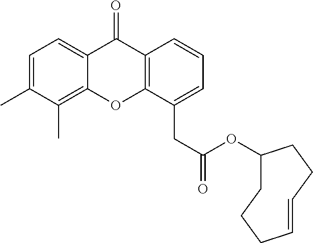 [SMILES: Cc1ccc2c(=O)c3cccc(CC(=O)OC4CC/C=C/CCC4)c3oc2c1C] |
| CdN | Thioester | TCO | 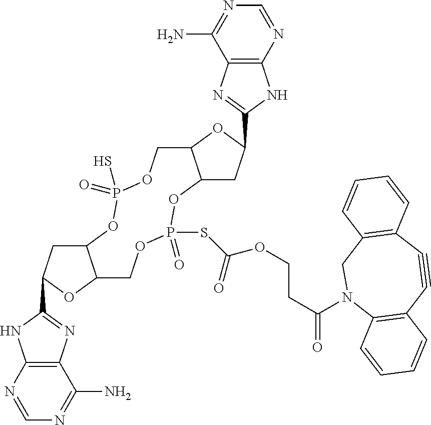 [SMILES: Nc1ncnc2[nH]c([C@H]3CC4OP(=O)(S)OCC5O[C@@H](c6nc7c(N)ncnc7[nH]6)CC5OP(=O)(SC(=O)OCCC(=O)N5Cc6ccccc6C#Cc6ccccc65)OCC4O3)nc12] |
| Temozolomide | Carbamate | DBC O | 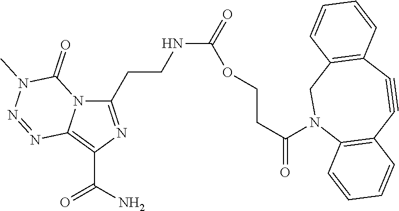 [SMILES: Cn1nnc2c(C(N)=O)nc(CCNC(=O)OCCC(=O)N3Cc4ccccc4C#Cc4ccccc43)n2c1=O] |
| Hydrocortisone | Ester | DBC O | 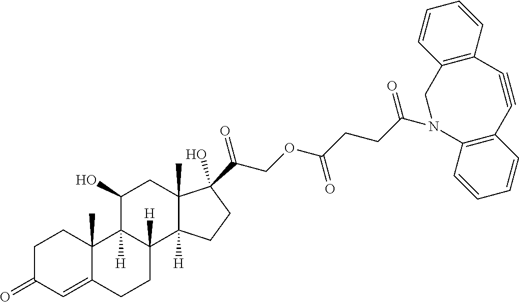 |
| Doxorubicin | Photolabile | DBC O | 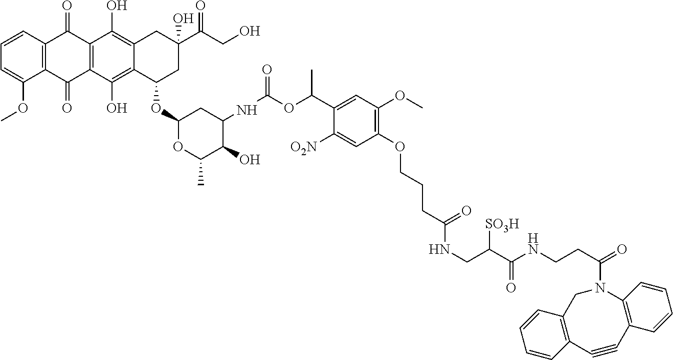 [SMILES: COc1cc(C(C)OC(=O)NC2C[C@H](O[C@H]3C[C@](O)(C(=O)CO)Cc4c(O)c5c(c(O)c43)C(=O)c3c(OC)cccc3C5=O)O[C@@H](C)[C@@H]2O)c([N+](=O)[O-])cc1OCCCC(=O)NCC(C(=O)NCCC(=O)N1Cc2ccccc2C#Cc2ccccc21)S(=O)(=O)O] |
| Docetaxel | Photolabile | DBC O | 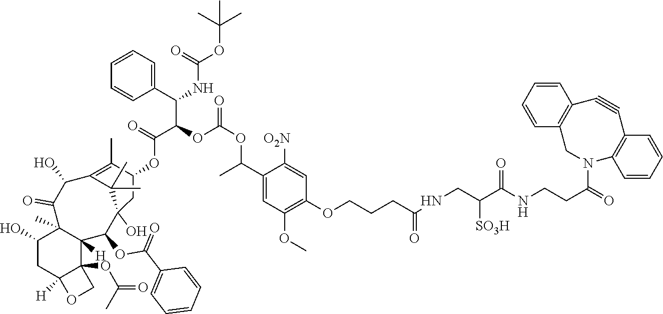 [SMILES: COc1cc(C(C)OC(=O)O[C@@H](C(=O)O[C@@H]2C[C@@]3(O)[C@@H](OC(=O)c4ccccc4)[C@@H]4[C@]5(OC(C)=O)CO[C@H]5C[C@H](O)[C@@]4(C)C(=O)[C@H](O)C(=C2C)C3(C)C)[C@@H](NC(=O)OC(C)(C)C)c2ccccc2)c([N+](=O)[O-])cc1OCCCC(=O)NCC(C(=O)NCCC(=O)N1Cc2ccccc2C#Cc2ccccc21)S(=O)(=O)O] |
| Doxorubicin | Photolabile | TCO | 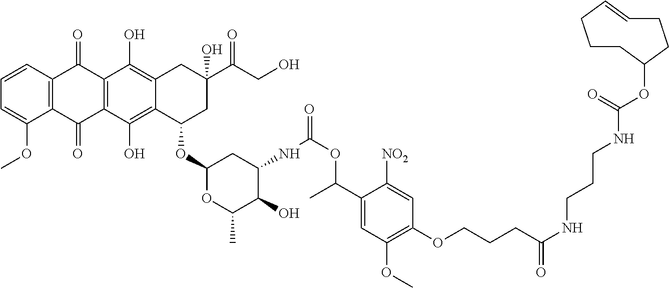 [SMILES: COc1cc(C(C)OC(=O)N[C@H]2C[C@H](O[C@H]3C[C@](O)(C(=O)CO)Cc4c(O)c5c(c(O)c43)C(=O)c3c(OC)cccc3C5=O)O[C@@H](C)[C@@H]2O)c([N+](=O)[O-])cc1OCCCC(=O)NCCCNC(=O)OC1CC/C=C/CCC1] |
| Docetaxel | Photolabile | TCO | 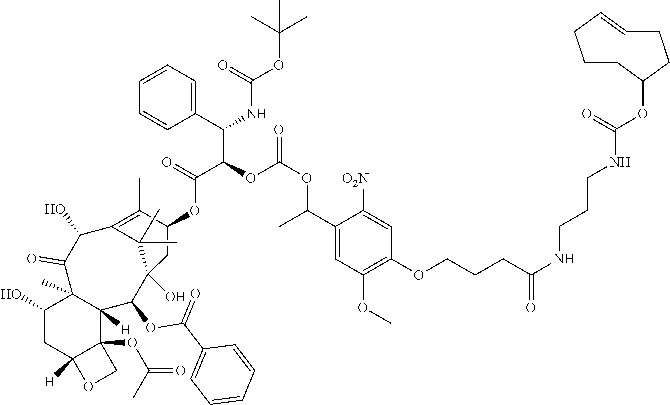 [SMILES: COc1cc(C(C)OC(=O)O[C@@H](C(=O)O[C@H]2C[C@@]3(O)[C@@H](OC(=O)c4ccccc4)[C@@H]4[C@]5(OC(C)=O)CO[C@@H]5C[C@H](O)[C@@]4(C)C(=O)[C@H](O)C(=C2C)C3(C)C)[C@@H](NC(=O)OC(C)(C)C)c2ccccc2)c([N+](=O)[O-])cc1OCCCC(=O)NCCCNC(=O)OC1CC/C=C/CCC1] |
| Doxorubicin | Click Cleavable | TCO | 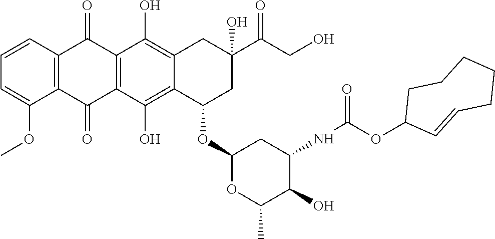 [SMILES: COc1cccc2c1C(=O)c1c(O)c3c(c(O)c1C2=O)C[C@@](O)(C(=O)CO)C[C@@H]3O[C@H]1C[C@H](NC(=O)OC2/C=C/CCCCC2)[C@@H](O)[C@H](C)O1] |
| Reciquimod | Click Cleavable | TCO | 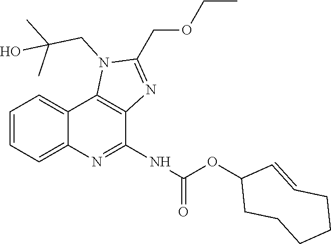 [SMILES: CCOCc1nc2c(NC(=O)OC3/C=C/CCCCC3)nc3ccccc3c2n1CC(C)(C)O] |

By way of non-limiting illustration, examples of certain embodiments of the present disclosure are given below.

### EXAMPLES

### Example 1: Targeted Intratumoral Therapy via NHS Ester Reactions Combined with Bioorthogonal Click Chemistry

Background

Local drug depots have been explored in order to reduce the off-target toxicities associated with systemic administration of pharmaceuticals. However, local depots deplete their drug storage and are unable to be refilled leading to a limited tumor suppression. Another hurdle for drug delivery is specific drug resistance due to the heterogenous makeup of malignancies. Being able to change drugs throughout treatment would increase a patient's chances of survival if resistance becomes a problem. Refillability of local depots would allow for clinicians to change drugs, doses, and frequency of administration allowing for temporal control over the individualized patient's therapy. Herein, a small molecule drug depot that can be injected intratumorally and be refilled systematically by click chemistry modified molecules is described.

Overview

This example relates to strategies for delivering drugs to diseased tissues. These strategies can translate the bolus, systemic administration of a therapy into the local and sustained drug delivery at only one site.

The strategies employ click chemistry to deliver a drug to a tissue of interest. Target tissue could be, for example, a tumor, an area of ischemia (heart attack, stroke), an area of local infection, an area of immunological organ injection, or other localized disease.

First, the tissue of interest is functionalized by injecting a reactive “click” molecule (referred to as a “Click-Target”) into the target tissue. The “Click-Target” is a bifunctional molecule including a first moiety that interacts (e.g., forms a covalent bond with) the target tissue, and a second moiety that can participate in a click reaction (a first “click motif”). By way of example, the first moiety can react to form a covalent bond with the proteins in the target tissue. These proteins could be part of the extracellular matrix or could be proteins on cells. The result is a tissue that is decorated with the “Click-Target” molecule, and thus display the second moiety that can participate in a click reaction (the first “click motif”). Optionally, the “Click-Target” con further include a linker (e.g., a bivalent linker) covalently joining the first moiety and the second moiety. When present, the linker may be a cleavable (e.g., hydrolysable) linker or a non-cleavable linker (if desired for a particular active agent and/or “Click Prodrug,” as discussed below).

Bioorthogonal “click” chemistry is then used to target drugs specifically to the “Click-Target.” Click chemistry refers to a class chemical reaction between two click groups that exhibit good yields, wide functional group tolerance, and are highly selective even in the presence of a complex mixture of biological molecules. These characteristics allow the click reactions to proceed even in vivo.

In a second step, a drug conjugate (referred to as a “Click Prodrug”) is administered to the subject. The “Click Prodrug” can include an active agent (e.g., a therapeutic, diagnostic, or prophylactic agent) conjugated to a second moiety that can participate in a click reaction (a second “click motif”) directly, or optionally through a linker (e.g., a bivalent linker). When present, the linker may be a cleavable (e.g., hydrolysable) linker or a non-cleavable linker (if desired for a particular active agent and/or “Click-Target”). The first “click motif” and the second “click motif” are selected such that they can react through a click reaction to form a covalent bond. Generally, the “Click Prodrugs” do not cross cell membranes and do not have activity (or toxicity) on their own. This can reduce systemic toxicity associated with administration of the active agent. However, the “Click Prodrug” does have the ability to “click” at the location of the “Click-Target,” thereby anchoring the active agent at the target tissue (e.g., the site of disease) that has been pre-labeled with the “Click Target.”

If desired for a particular application, the “Click Prodrug” can include an active agent (e.g., a therapeutic agent) conjugated to a second moiety that can participate in a click reaction (a second “click motif”) through a cleavable (e.g., hydrolysable) linker. In these cases, once the “Click Prodrug” has been bound to the “Click-Target,” the cleavable linker tethering the active agent to the “Click-Target” can be cleaved (e.g., via hydrolysis), releasing the active agent into the target tissue. Based on the nature of the cleavable linker, the cleavage rate (and by extension the drug delivery profile of the active agent) can be tuned to provide controlled deliver of the active agent.

In other cases, the “Click Prodrug” can include an active agent conjugated to a second moiety that can participate in a click reaction (a second “click motif”) directly or through a non-cleavable linker. In these cases, once the “Click Prodrug” has been bound to the “Click-Target,” the active agent remains bound to the “Click Target,” assuming the “Click-Target” does not include a cleavable linker. This can allow for an active agent (e.g., a therapeutic agent such as a radionuclide, or a diagnostic agent such as an imaging agent) to be retained (i.e., not released) into the target tissue.

These strategies can be used to efficiently target active agents to subcutaneous and intratumoral sites within a subject, including within the brain.

Example Systems for Drug Delivery

An example “Click-Target” (also referred to as a small molecule depot) incorporates an N-Hydroxylsuccinimide (NHS) ester which can rapidly form covalent bonds with primary amines. Notably, sulfo-NHS esters can be readily soluble in water, which gets rid of the need for organic solvents. To create a depot that can be injected in the body, an ester was used that takes advantage of the presence of upregulated collagen expression in fibrous tumors such a pancreatic cancer. The extracellular matrix of tumors includes collagen that has the amino acid lysine consisting of a primary amine which would react readily with an ester molecule. Pancreatic tumors have a threefold increase in the quantity of collagen allowing for more intratumoral binding sites to anchor the injected small molecule drug depot. Levels of 12.5 g hydroxyproline/100 g protein have been observed for collagen in wet tissue calculations, so an estimation of 12.5% of collagen being hydroxyproline was used. Dividing 6.84 ug 4-Hydroxyproline/mg wet pancreatic cancer tissue measured from human pancreatic tumors by 0.125 gave 54.72 ug collagen/mg wet pancreatic cancer tissue compared to 18.24 ug collagen/mg wet healthy pancreas tissue. A sulfo-NHS ester conjugate binds to the extracellular matrix of tumors and diffuse spherically outwards from the syringe infusion. The ester's reaction with the extracellular matrix amines anchors the click motifs inside the tumor to act as local depot to capture and release active drugs over weeks at a time. This technique can be used to, for example, treat unresectable tumors.

In this example, the sulfo-NHS ester conjugate is modified with a click chemistry azide motif. Copper free click chemistry reactions were first described by Sharpless in 2001 as “spring loaded” reactions that have high yields, large thermodynamic driving forces, inoffensive byproducts, and performed under simple reaction conditions. Specifically, a copper free click chemistry called the Staudinger 1,3-dipolar cycloaddition reaction between an azide and cyclooctene in vivo has been shown to track specific protein localizations using fluorescent peptides without apparent toxicity. Brudno et al. (Brudno, Y. et al. In vivo targeting through click chemistry. ChemMedChem 10, 617-620 (2015), which is hereby incorporated by reference in its entirely) has demonstrated the homing of prodrugs to alginate gels implanted at disease sites using the Cu free click chemistry. This allows for multiple refills of a depot to allow a physician to temporally control treatment, change doses, or drug types. Utilizing the in vivo bioorthogonal properties of the Staudinger reaction, the system described herein can be filled with inert prodrugs for gradual release over weeks at a time with only a small molecule injection at a tumor site.

These strategies can be used for the treatment for any fibrous tumor or disease that can be reached surgically with a syringe needle tip. Analogous strategies can be used to load medical devices (e.g., stents) with active agents. For example, medical devices can be contacted with a suitable “Click-Target” (in vivo or ex vivo) to decorate the surface of the medical device with a first click motif. Following implantation, a “Click Prodrug” can then be administered to tether an active agent to the medical device. If a cleavable linker is present (in the “Click-Target” or “Click Prodrug”), the active agent can then be eluted from the medical device over time. This strategy can be used for example, to provide medical devices, such as stents, that can loaded (and reloaded) with antibiotics for infection treatments.

Materials and Methods

COMSOL Modeling. Predictions of the azide-s-nhs ester diffusion were performed in the COMSOL finite element analysis software coupling Darcy's law, reaction engineering, and transport of a reactive species modules.

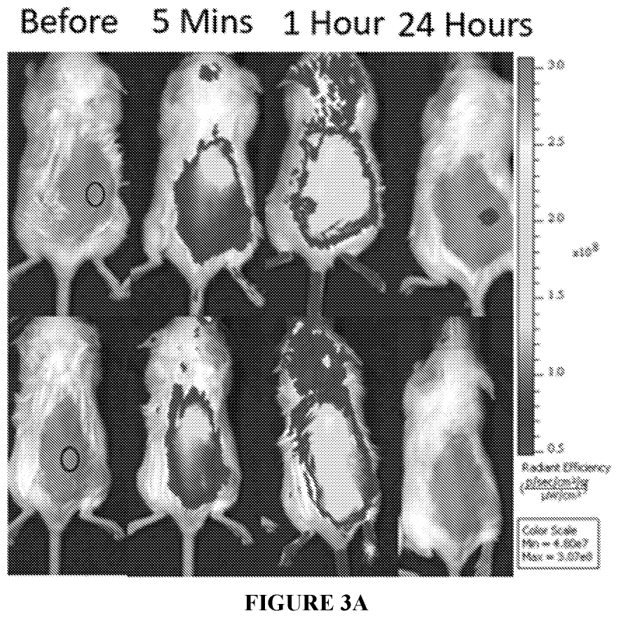

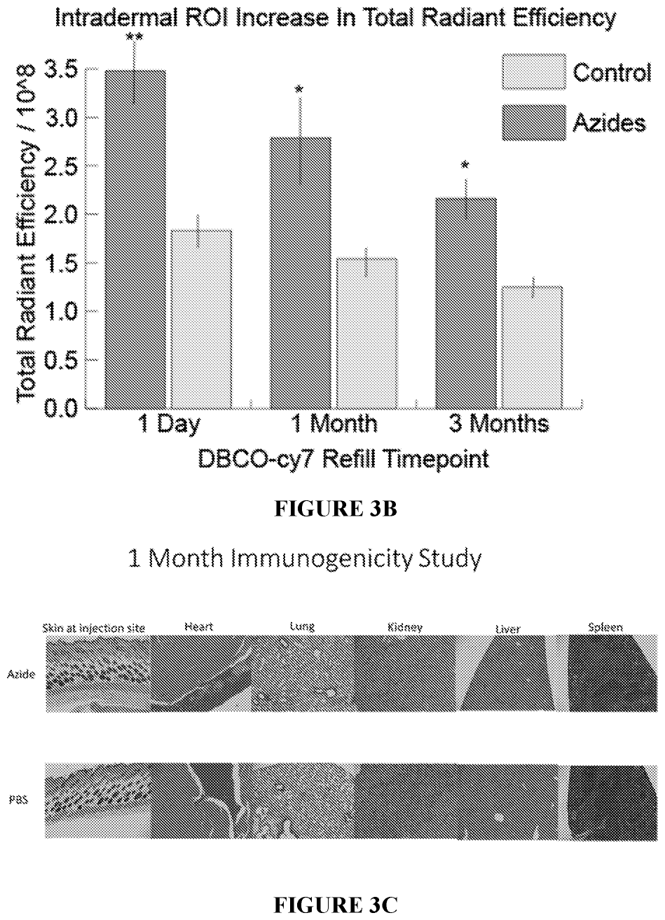

Intradermal Refillablitiy. CD1 mice from Charles River were injected intradermally on the dorsal flank with 50 ul of 0.2M azide-s-nhs ester (synthesized in Pierce lab December 2017) or PBS. Mice were then imaged to get a before picture and then injected retro orbitally with DBCO-cy7 (lot number) before taking another image on the in vivo IVIS Spectrum Imager at 5 mins, 1 hour, and 24 hours. The mice were then injected with DBCO-cy7 at 1 month, 3 months, and (soon) 6 months to show the long-term refillablity of the system. The data was analyzed using total radiant efficiency measured within regions of interest (ROI) over the intradermal injection area in the IVIS imaging software live image. The total radiant efficiencies plotted in FIG. 3B were the values obtained 24 hours after injection subtracting the before fluorophore injection radiant efficiency measurements.

Allograft Pancreatic Tumor. KPC 4662 pancreatic tumor cells (5e5) were injected in a 1:1 ratio with PBS:Matrigel(Matrigel Matrix 354234, lot 7205011) into the subcutaneous dorsal flanks of CD57B16 mice. Mice grew tumors over a week and then were injected with 50 uL of 200 mM 3-Azidopropionic Acid Sulfo-NHS ester (made from Pierce lab) or 1× PBS intratumorally.

IVIS imaging (Camera #11219, DW434) was performed right before 100 ul of 50 mg/ml DBCO-cy7 retro orbital injections and imaged 24 hours afterwards when most of the unbound DBCO-cy was cleared from systemic circulation. For the refill dose experiments 9-week-old albino c57b16's from Envigo were used. For all other tumor refills, 9-week-old regular c57s from Envigo were used.

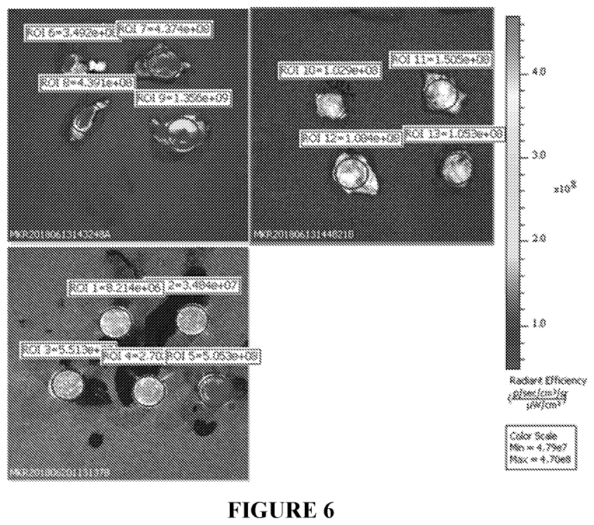

Dose Captured. 8 mice were similarly injected with KPC 4662 tumors and allowed to grow over a week until injection of sNHS(N=4) or PBS(N=4). Injection and imaging was performed as described above on the IVIS before and 24 hours after DBCO-cy7 IV injection. Tumors were then explanted imaged to compare to a linear regression alginate gels ROI's that contained the same dose injected IV to obtain a percentage of cy7 captured at the tumor site. (See FIG. 6)

Light Sheet Microscopy. KPC 4662 tumors were grown in the dorsal flanks of mice for 1 week and then injected with 0.2M azide-s-NHS ester or PBS. Perfusion was performed soon after (˜15 minutes) with PBS followed by 4% formalin using the gravity perfusion system. To clear the tissue, perfused tumors were immediately flash frozen in OCT embedding medium (lot number 4298, exp June 2021) to preserve morphology. Tumors were then placed in 4% formaldehyde for an hour to remove OCT, followed by 24 hours at 4° C. overnight for fixation, and then the iDISCO protocol was followed until the tumors were cleared in DBE. Pictures of the cleared tumor were taken with an Utlramicroscope 2. Images analyzed on Imaris 9.0 software.

Brain Refills. For anesthesia, 1-5% isoflurane was administered before procedures as well as continuously through the stereotaxic setup, and 0.5% lidocaine HCL administered subcutaneous under the incision site prior to surgery. 10-week-old CD1 mice were mounted on a kopf stereotaxic head stage using ear bars and a 5 μl Hamilton syringe placed in needle fixture. For surgery, the area to be incised on the head was shaved and washed with 70% alcohol. An incision with a #10 blade was made from posterior to anterior (start of skull to between eyes) and skin pulled away from incision using sterile Q-tips to expose the skull. The syringe needle was adjusted so the tip was at Bregma, position of the stereotaxic device recorded, and then moved to the desired calculated position where a marking will be made with a surgical marker. The experimental coordinates were −2.5M/L, −1A/P, −3D/V. A small hand drill was then used to make a 1-2 mm size hole in the skull for the injection needle. Once the hole in the skull was roughly 1-2 mm wide, the needle was adjusted to injection position and slowly lowered to desired depth from the skull opening. 2 uL of 0.5M azide-sulfo-NHS dissolved in sterile PBS or sterile PBS was be slowly injected into the brain over 15 minutes. Once complete, the mouse skin was joined by VetBond skin glue and monitored post-surgery. Each mouse was then left to heal for at least one week.

Once healed, a fluorophore imagine agent was administered and the mice were imaged. For fluorophore infusions, an IV was inserted into the tail vein via a 28 G catheter with a 100 μL of mannitol filled syringe that has been filtered through a 0.45 mm diameter filter (25%, for IV use only). Concurrently with the injection of mannitol, 100 μl of far red labeled DBCO (for azide-sNHS injected) was administered to each mouse through the same catheter in the tail vein. Imaging on the IVIS spectrum was performed at 24 hours after drug injection for each timepoint. Live animal imaging on the IVIS spectrum was used to evaluate the intensity of fluorescent linker molecules reaching the gel to determine potential drug homing efficiency to intracranial depots.

If the mouse was not utilized for IVIS imaging, we also perfused the azide-sNHS injected mice with 10 ml of saline followed by 10 ml of 4% formaldehyde through a 27 G catheter inserted into the apex of the heart to fix the brain tissue. The mouse brains were excised and put through a similar fixing, staining, and clearing process (iDISCO) as described for the tumors.

Results and Discussion

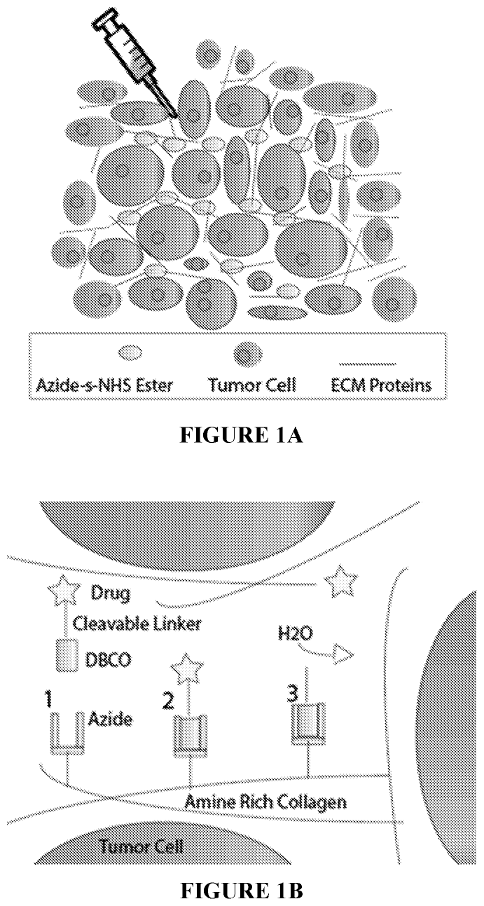

FIGS. 1A and 1B schematically illustrate methods for drug delivery to a target tissue. As shown in FIG. 1A, a target tissue is first exposed to a “Click-Target,” decorating the tissue with a moiety that can participate in a click reaction. In this example, the “Click-Target” includes an amine-reactive NHS (N-hydroxysuccinimide) ester or sulfo-NHS ester and an azide moiety. The amine-reactive NHS (N-hydroxysuccinimide) ester or sulfo-NHS ester reacts with amines present in extracellular matrix (ECM) proteins to form a covalent bond. As shown in FIG. 1B, following treatment with the “Click-Target,” ECM proteins within the target tissue display azide moieties covalently tethered to the ECM proteins (1). The target tissue is then contacted with a “Click Prodrug.” In this example, the “Click Prodrug” includes an active agent (e.g., a therapeutic, diagnostic, or prophylactic agent) conjugated to a second moiety that can participate in a click reaction (a second “click motif”) through a cleavable bivalent linker. In this example, the second click motif is a diarylcyclooctyne moiety, which can participate in a click reaction with the azide moieties tethered to the ECM proteins to form a covalent bond. As a result, the active agent becomes covalently tethered to the ECM proteins within the target tissues (2). Subsequently, the cleavable bivalent linker in the “Click Prodrug” is cleaved via hydrolysis, releasing the active agent into the target tissue over time (3).

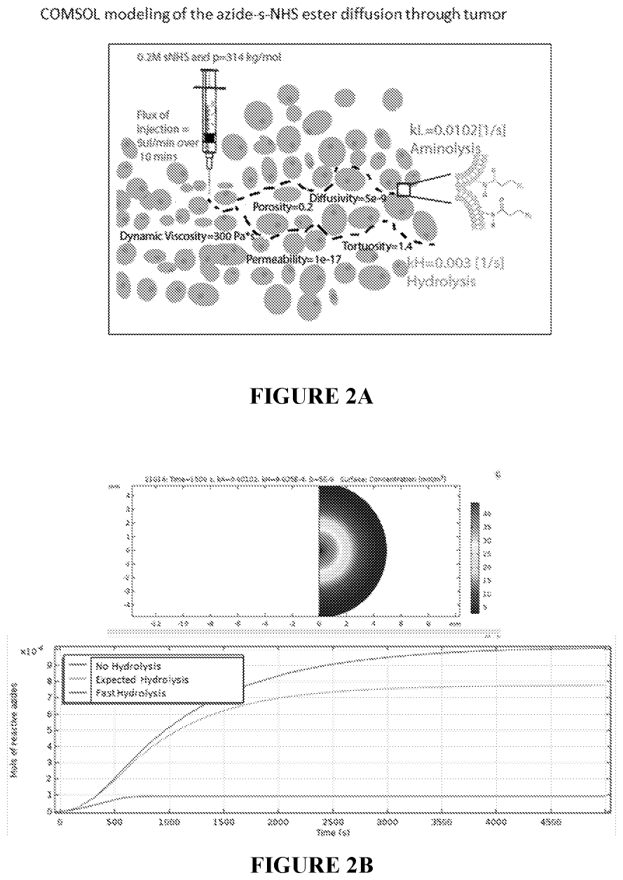

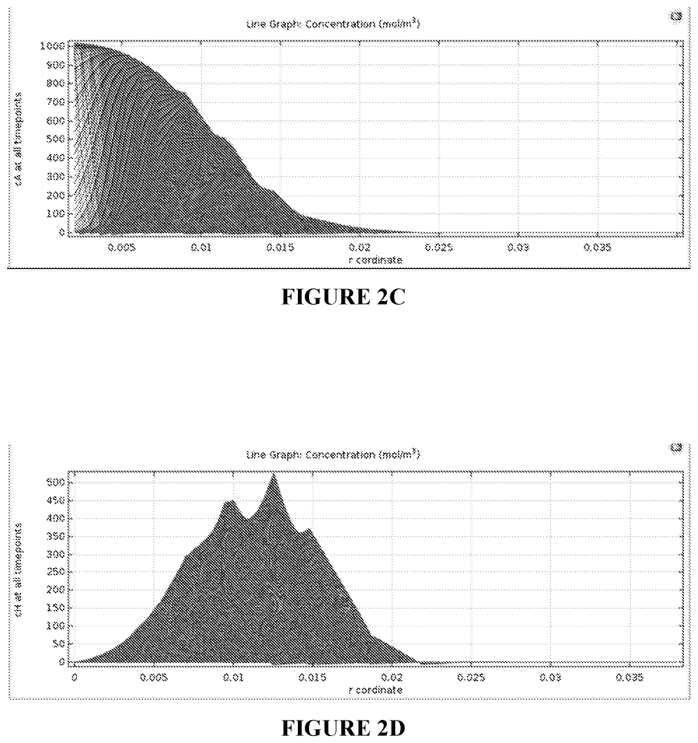

COMSOL modeling was used to predict the formation of an azide-based depot upon injection of a “Click-Target” into a tumor. FIGS. 2A-2D illustrate the parameters and results of COMSOL modeling of the diffusion of an example “Click-Target” through a tumor. Assuming an injection of a total volume of 50 μL of 0.2 M “Click-Target” over a period of 10 minutes, the modeling provided an expected aminolysis rate of 0.0102 [1/s], an expected hydrolysis rate of 0.003 [1/s], and an expected diffusivity of 5×10⁻⁹.

As shown in FIG. 2C, the amount of “Click-Target” in the radial coordinates away from in infusion site can be calculated. Each line represents the concentration of “Click-Target” a different timepoint. Integration of this curve provides the total amount of reactive azides. As shown in FIG. 2D, the amount of hydrolyzed azide that does not become reactive (cannot bind with DBCO molecules) can similarly be determined.

As shown in FIGS. 3A and 3B, intradermal administration of DBCO-cy7 (a model “Click Prodrug”) was used to demonstrate the ability to target the reactive azides for repeated long-term dosing of an active agent. Cyanine7 DBCO is a NIR fluorescent dye with cycloalkyne moiety for the conjugation with azides by means of copper-free, strain promoted alkyne azide cycloaddition (spAAC). FIG. 3A shows the localization of the DBCO-cy7 (“Click Prodrug”) near the site where the “Click-Target” injection (indicated by the circle superimposed in the before images. FIG. 3B shows subsequent reloading of the depot at 1 day, 30 days, and 90 days.

FIG. 3C shows histology performed one month following injection of the azide-s-nhs ester. H&E slides of mouse skin and tissues from 8 mice were analyzed to determine local or distal effects of an injected depot material. All tissues were evaluated by light microscopy and were graded as: “-”=no changes observed in this tissue for any mouse; “0”=a particular change not present; “1”=change is present at slight/minimal severity; “2”=mild severity, “3”=moderate severity; “4” marked severity; “5”=severe. A grade of 1 represents the minimal detectable change and a 5 represents a change judged to be essentially as severe as possible. Minimal histologic alterations were noted in the skin of one mouse and the livers of 3 mice (each scored as a 1). All others scored a 0. Mouse #7, Group B, has a very small focus of slightly increased numbers of neutrophils and macrophages in the hypodermis. A few of the macrophages contained dull reddish-brown isotropic pigment which could be lipofuscin, or possibly hemosiderin or other material. This area may be the site of injection. Mouse #2, Group A, has a single small aggregate of macrophages in a hepatic sinusoid. Mice #6 and #8, both Group B, have similarly sized small clusters of leukocytes, which include a few neutrophils in addition to macrophages (probably an earlier stage of the same process). Hepatic aggregates of leukocytes in these 3 mice likely represent a progressive response to blood-borne inflammatory particles and are frequently observed in both manipulated and control mice. When infrequent and small, as in these mice, they are generally considered to be of no research significance or biologic consequence.

No differences were noted between the two groups.

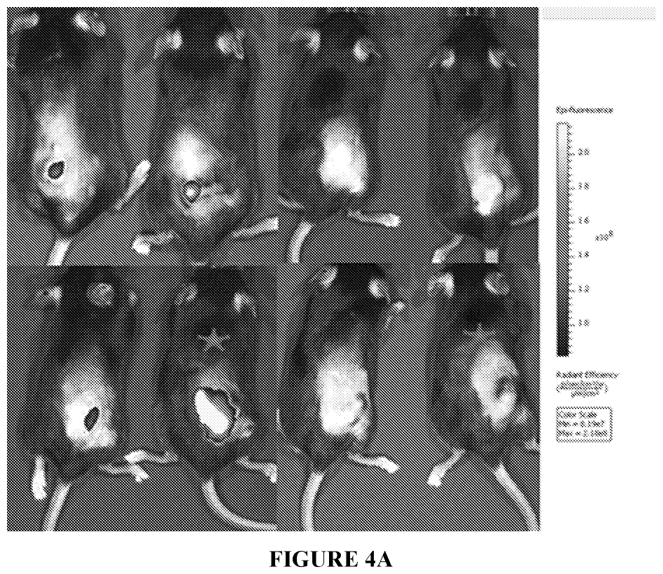

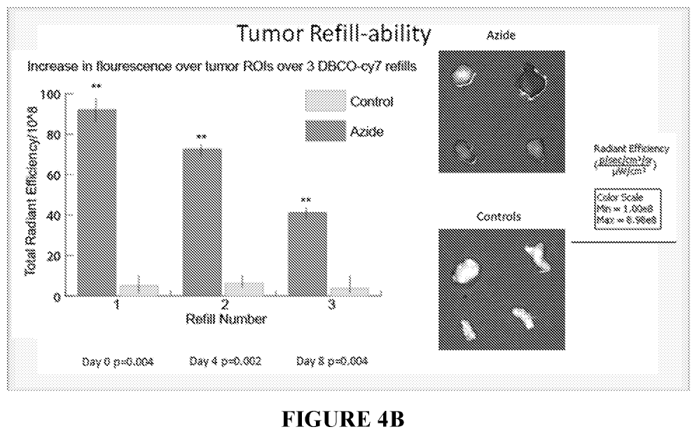

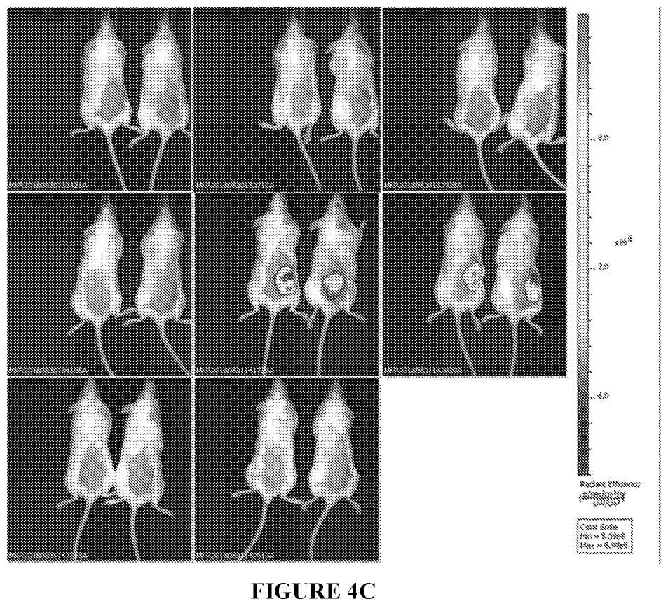

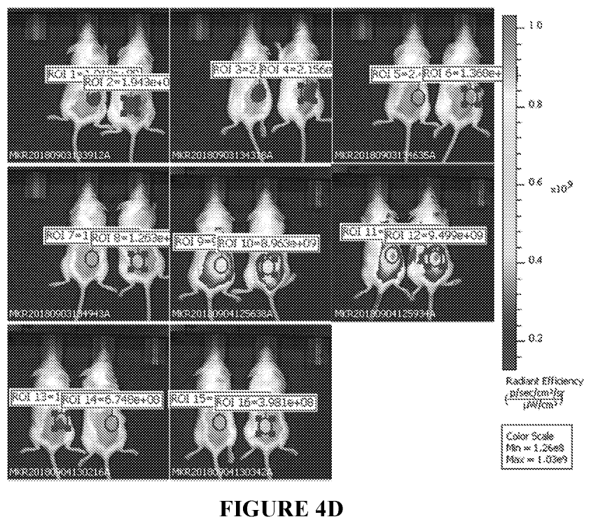

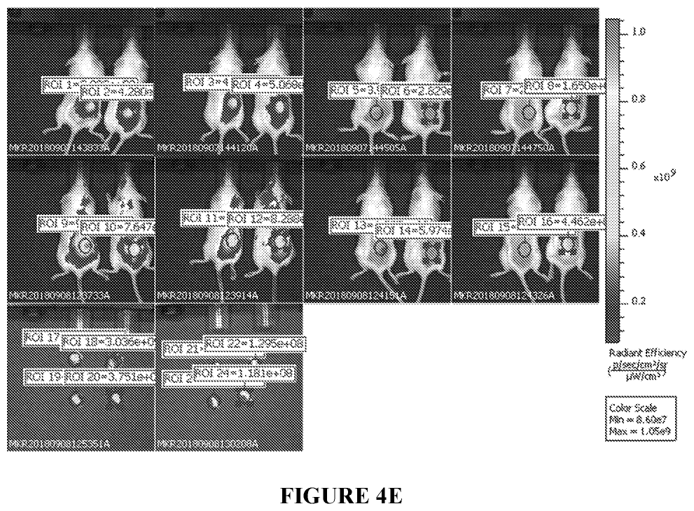

FIG. 4A shows a tumor treated with the azide-s-nhs ester (“Click-Target”) before administration of DBCO-cy7 (a model “Click Prodrug”), and 24 hours after administration of a first dose of DBCO-cy7 (the model “Click Prodrug”). As shown in FIG. 4B, subsequent administrations of DBCO-cy7 can be used to refill the azide depot. FIG. 4C shows the localization of cy7 at the azide-decorated tissue 24 hours after injection. The top and middle left images show a mouse treated with the “Click-Target” (azide sNHS) before injection of DBCO-cy7 (the model “Click Prodrug”); the top right and middle left images show a control mouse treated with PBS before injection of DBCO-cy7 (the model “Click Prodrug”); the middle and middle right images show a mouse treated with the “Click-Target” (azide sNHS) after injection of DBCO-cy7 (the model “Click Prodrug”); and the bottom two images show a control mouse treated with PBS after injection of DBCO-cy7 (the model “Click Prodrug”).

The ROIs over the mouse tumors produced the following increases in fluorescence over tumor sites following the first administration of DBCO-cy7 (the model “Click Prodrug”) are shown in the table below.

| | Total Radiant Efficiency | |
|---|---|---|
| | Measured within Regions | Average Radiant |
| Mouse | of Interest (ROI) | Efficiency |
| Azide sNHS Mouse 1 | 9.29 × 10⁹ | 9.21 × 10⁹ |
| Azide sNHS Mouse 2 | 8.87 × 10⁹ | |
| Azide sNHS Mouse 3 | 9.86 × 10⁹ | |
| Azide sNHS Mouse 4 | 8.81 × 10⁹ | |
| Control Mouse 1 | 8.93 × 10⁸ | 5.20 × 10⁸ |
| Control Mouse 2 | 4.76 × 10⁸ | |
| Control Mouse 3 | 4.10 × 10⁸ | |
| Control Mouse 4 | 3.01 × 10⁸ | |

FIG. 4D shows the localization of cy7 at the azide-decorated tissue 24 hours after the second injection. The top and middle left images show a mouse treated with the “Click-Target” (azide sNHS) before the second injection of DBCO-cy7 (the model “Click Prodrug”); the top right and middle left images show a control mouse treated with PBS before the second injection of DBCO-cy7 (the model “Click Prodrug”); the middle and middle right images show a mouse treated with the “Click-Target” (azide sNHS) after the second injection of DBCO-cy7 (the model “Click Prodrug”); and the bottom two images show a control mouse treated with PBS after the second injection of DBCO-cy7 (the model “Click Prodrug”). The ROIs over the mouse tumors produced the following increases in fluorescence over tumor sites following the second administration of DBCO-cy7 (the model “Click Prodrug”) are shown in the table below.

| | Total Radiant Efficiency | Average | |
|---|---|---|---|
| | Measured within Regions | Radiant | Standard |
| Mouse | of Interest (ROI) | Efficiency | Deviation |
| Azide sNHS | 7.57 × 10⁹ | 7.26 × 10⁹ | 2.46 × 10⁸ |
| Mouse 1 | | | |
| Azide sNHS | 7.02 × 10⁹ | | |
| Mouse 2 | | | |
| Azide sNHS | 7.11 × 10⁹ | | |
| Mouse 3 | | | |
| Azide sNHS | 7.34 × 10⁹ | | |
| Mouse 4 | | | |
| Control Mouse 1 | 1.19 × 10⁹ | 6.34 × 10⁸ | 3.89 × 10⁸ |
| Control Mouse 2 | 5.38 × 10⁸ | | |
| Control Mouse 3 | 5.41 × 10⁸ | | |
| Control Mouse 4 | 2.72 × 10⁸ | | |

FIG. 4E shows the localization of cy7 at the azide-decorated tissue 24 hours after the third injection. The top and middle left images show a mouse treated with the “Click-Target” (azide sNHS) before the third injection of DBCO-cy7 (the model “Click Prodrug”); the top right and middle left images show a control mouse treated with PBS before the third injection of DBCO-cy7 (the model “Click Prodrug”); the middle and middle right images show a mouse treated with the “Click-Target” (azide sNHS) after the third injection of DBCO-cy7 (the model “Click Prodrug”); and the bottom two images show a control mouse treated with PBS after the third injection of DBCO-cy7 (the model “Click Prodrug”). The ROIs over the mouse tumors produced the following increases in fluorescence over tumor sites following the third administration of DBCO-cy7 (the model “Click Prodrug”) are shown in the table below.

| | Total Radiant Efficiency | |
|---|---|---|
| | Measured within Regions | Average Radiant |
| Mouse | of Interest (ROI) | Efficiency |
| Azide sNHS Mouse 1 | 5.77 × 10⁹ | 4.12 × 10⁹ |
| Azide sNHS Mouse 2 | 3.37 × 10⁹ | |
| Azide sNHS Mouse 3 | 4.13 × 10⁹ | |
| Azide sNHS Mouse 4 | 3.22 × 10⁹ | |
| Control Mouse 1 | 6.44 × 10⁸ | 3.92 × 10⁸ |
| Control Mouse 2 | 3.15 × 10⁸ | |
| Control Mouse 3 | 3.30 × 10⁸ | |
| Control Mouse 4 | 2.81 × 10⁸ | |

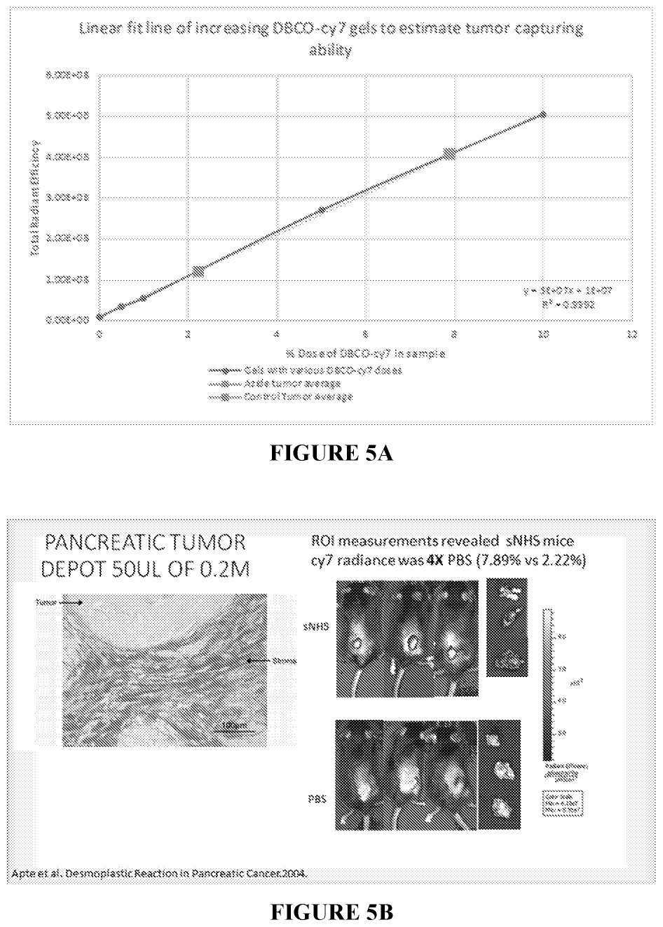

Alginate gels were synthesized with food grade alginate dissolved in 1.22M CaSO₄ in PBS overnight. Each gel was crosslinked was then crosslinked with different doses (0, 0.5, 1, 5, and 10%) of the DBCO-cy7 dose that was administered to mice in the trials above. As shown in FIG. 5A, a linear trendline was made to predict the dose of “Click Prodrug” caught at the depot site.

FIG. 5B shows a histology image of a pancreatic tumor following delivery of a dose of “Click Prodrug.” ROI measurements revealed that radiance was four times greater in mice treated with the “Click-Target” as compared. FIG. 6 shows a histology image from cryostat slices of tumors stained with DAPI and DBCO-cy3. The study employed three tumors injected with a “Click Target” and one control.

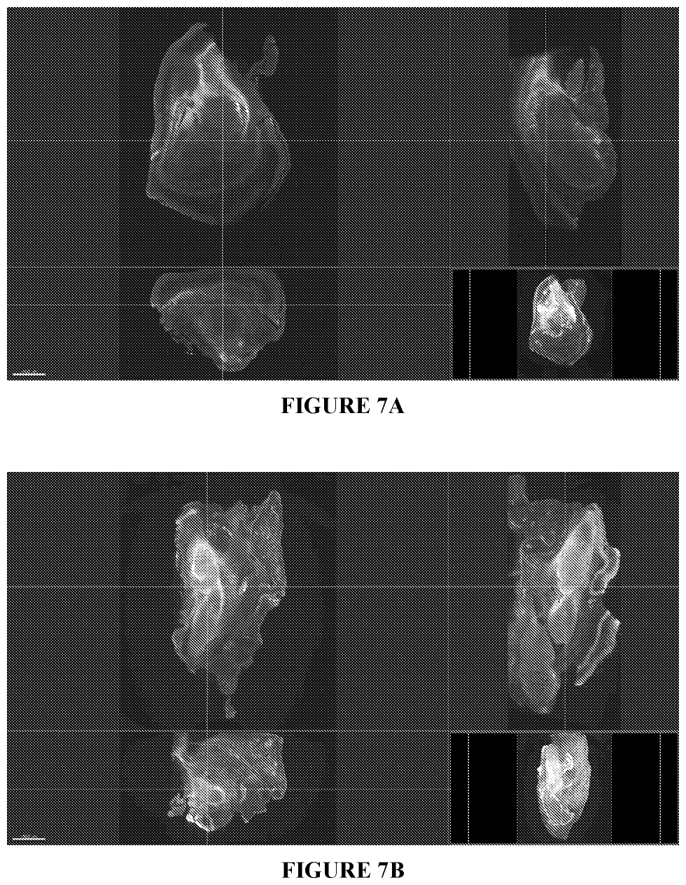

FIGS. 7A and 7B show an explanted tumor with no dbco-cy7 refill injection put through iDisco clearing, stained with DBCO-AF647, and imaged on a light sheet microscope. FIG. 7A shows the tumor infused with a “Click-Target” and “Click Prodrug.” FIG. 7B shows a control.

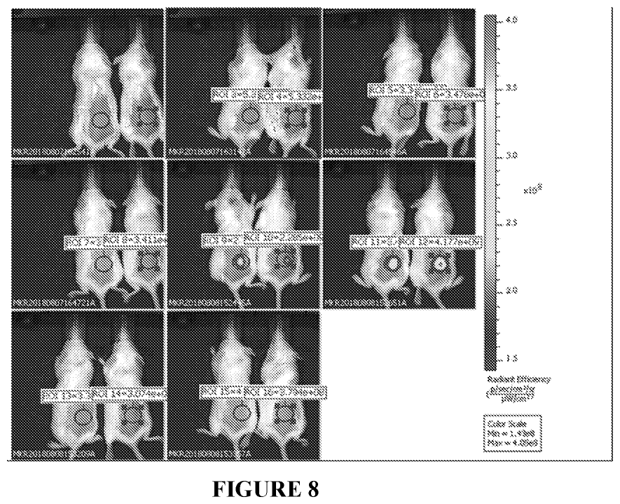

In another experiment, a different “Click-Target” (methyl tetrazine-sNHS ester) and model “Click Prodrug” (Cy5-TCO; a cyanine dye containing a trans-cyclooctene moiety) were used to demonstrate the applicability of this strategy to “Click-Targets” and “Click Prodrugs” containing other click motifs. 50 μL of 0.05M methyl tetrazine-sNHS ester was dissolved in sterile PBS, and injected intradermally into the dorsal flanks of mice CD1 Charles river mice. The first four images in FIG. 8 are pictures of the four mice treated with the “Click-Target” (tetrazine-sNHS) and the four control mice injected with PBS. The last 4 images in FIG. 8 show the fluorescence observed 24 hours after injection of the “Click Prodrug” (TCO-cy5). The IVIS was adjusted for this cy5 fluorophore. As shown in FIG. 8, the “Click Prodrug” was effectively bound at the site of “Click-Target” injection. This experiment was also performed at 1 week with similar results

The total increase in fluorescence after the TCO-cy5 injection (at 1 day) over the intradermal injection ROI's (total radiant efficiency measurement) subtracting the before tco-cy5 image injection value are included below.

| | Total Radiant Efficiency | Average | |
|---|---|---|---|
| | Measured within Regions | Radiant | Standard |
| Mouse | of Interest (ROI) | Efficiency | Deviation |
| Tetrazine-sNHS | 2.40 × 10⁹ | 2.72 × 10⁹ | 7.4 × 10⁸ |
| Mouse 1 | | | |
| Tetrazine-sNHS | 1.92 × 10⁹ | | |
| Mouse 2 | | | |
| Tetrazine-sNHS | 2.90 × 10⁹ | | |
| Mouse 3 | | | |
| Tetrazine-sNHS | 3.64 × 10⁹ | | |
| Mouse 4 | | | |
| Control Mouse 1 | 2.10 × 10⁷ | 3.87 × 10⁷ | 1.4 × 10⁷ |
| Control Mouse 2 | 4.04 × 10⁷ | | |
| Control Mouse 3 | 5.49 × 10⁷ | | |
| Control Mouse 4 | 3.83 × 10⁷ | | |

The total increase in fluorescence after the TCO-cy5 injection (at 1 week) over the intradermal injection ROI's (total radiant efficiency measurement) subtracting the before tco-cy5 image injection value are included below.

| | Total Radiant Efficiency | Average | |
|---|---|---|---|
| | Measured within Regions | Radiant | Standard |
| Mouse | of Interest (ROI) | Efficiency | Deviation |
| Tetrazine-sNHS | 1.57 × 10⁹ | 1.25 × 10⁹ | 2.5 × 10⁸ |
| Mouse 1 | | | |
| Tetrazine-sNHS | 1.31 × 10⁹ | | |
| Mouse 2 | | | |
| Tetrazine-sNHS | 1.01 × 10⁹ | | |
| Mouse 3 | | | |
| Tetrazine-sNHS | 1.12 × 10⁹ | | |
| Mouse 4 | | | |
| Control Mouse 1 | 3.81 × 10⁷ | 6.25 × 10⁷ | 1.7 × 10⁷ |
| Control Mouse 2 | 6.89 × 10⁷ | | |
| Control Mouse 3 | 6.68 × 10⁷ | | |
| Control Mouse 4 | 7.60 × 10⁷ | | |

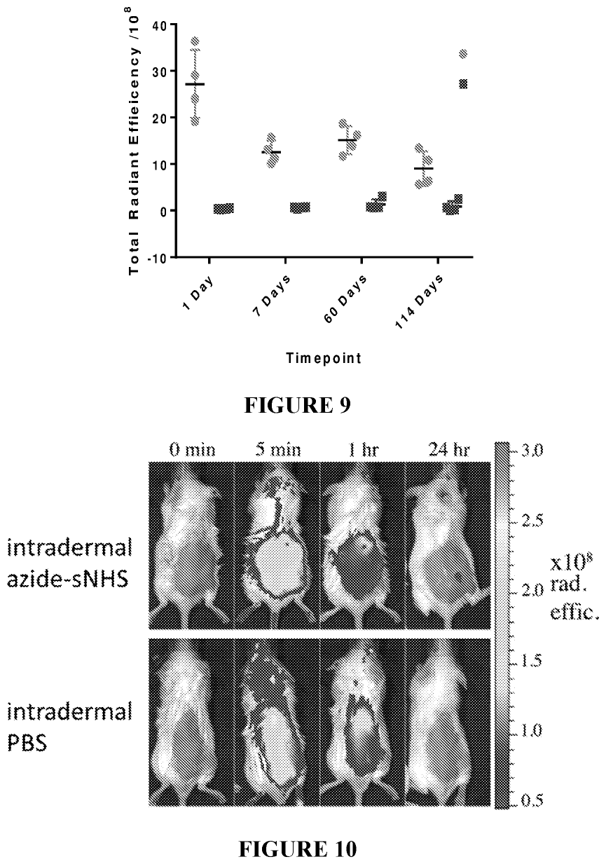

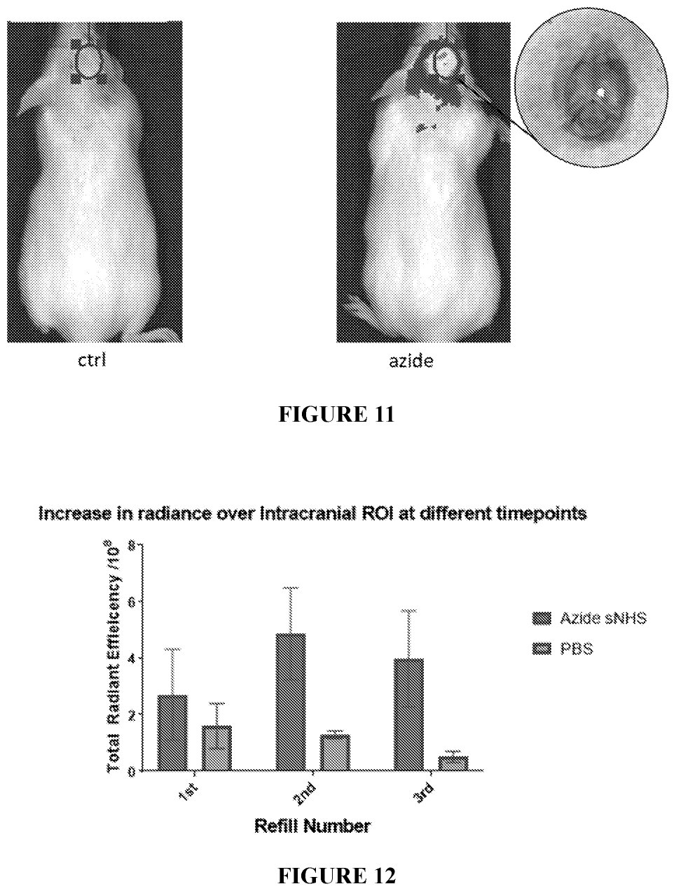

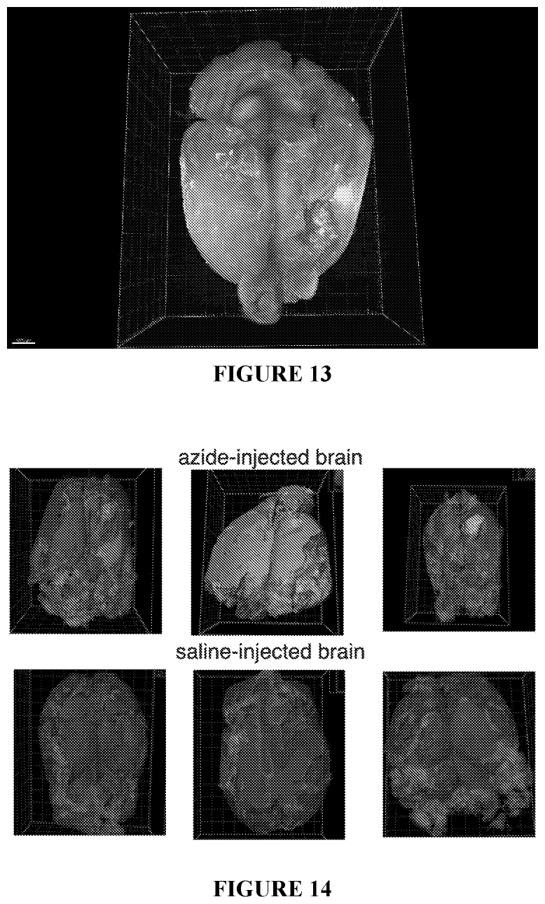

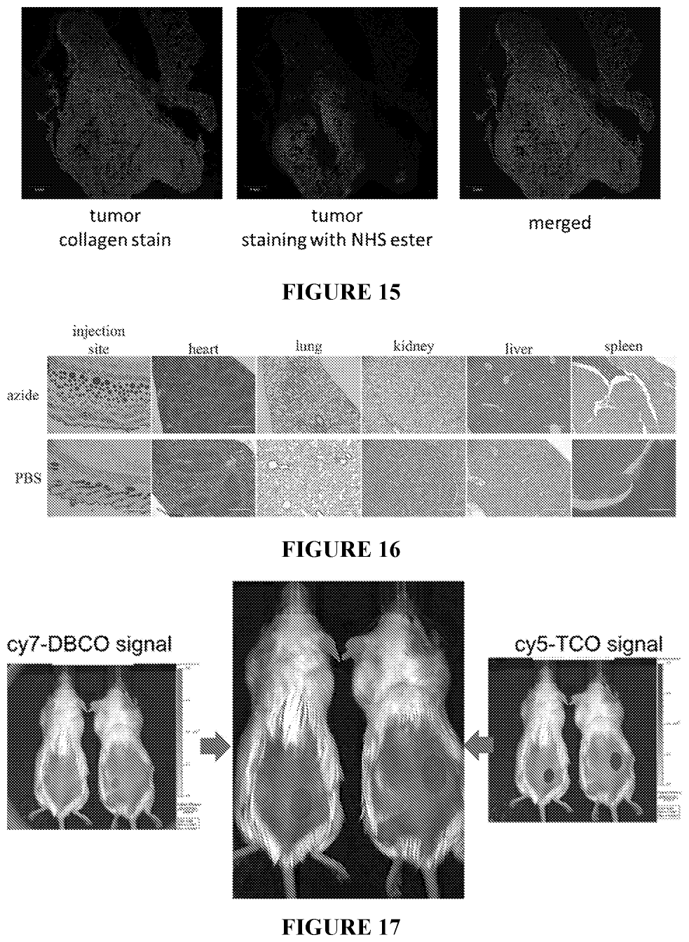

FIGS. 9-13 demonstrate the efficacy of a locally administered capture agent (a click target) to capture an intravenously injected model click prodrug (a click motif-labeled fluorophore). FIGS. 14-15 show results obtained following local administration of the model click prodrug. As shown in FIG. 16, injection in tissue did not induce an immune response locally at the site of injection.

FIG. 17 demonstrates that azide-sNHS ester depots are mutually compatible with tetrazine-sNHS ester depots for spatial separation of different regiments. As such, two different chemistries could be used to simultaneously deliver two agents to different locales within a subject. 50 μL of methyltetrazine sNHS (right, 0.05M) or azide-sNHS (left, 0.05M) was injected intradermally on the dorsal flank of 4 mice. 200 μL of 1:1 DBCO-Cy7/TCO-Cy5 were injected i.v. and imaged on the IVIS after 24 hours under the Cy7 and Cy5 filters.

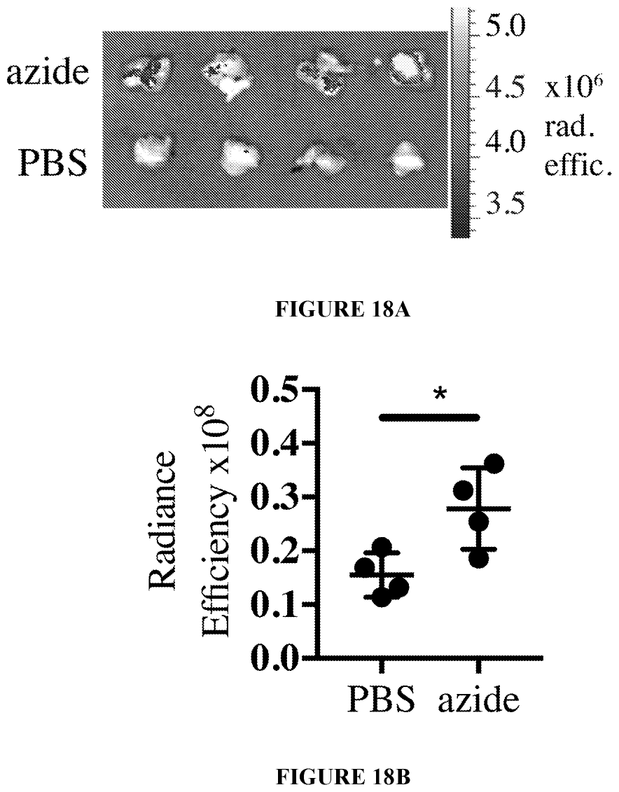

FIGS. 18A-18B demonstrated the compatibility of this system with antibody active agents. FIGS. 18A-18B show the click-specific capture of checkpoint blockade PD-1 antibodies at pancreatic tumor sites. FIG. 18A show extracted azide-sNHS infused tumors (top row) and PBS (bottom row) 24 hours after i.v. dosing with DBCO- and Cy7-conjugated anti-PD1 antibody. FIG. 18B show the quantification of radiant efficiency over tumor and underlying carcass ROI's 24 hours after i.v. DBCO-Cy7-antibody administration.

The compositions, systems, and methods of the appended claims are not limited in scope by the specific devices, systems, and methods described herein, which are intended as illustrations of a few aspects of the claims. Any devices, systems, and methods that are functionally equivalent are intended to fall within the scope of the claims. Various modifications of the devices, systems, and methods in addition to those shown and described herein are intended to fall within the scope of the appended claims. Further, while only certain representative devices, systems, and method steps disclosed herein are specifically described, other combinations of the devices, systems, and method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than where noted, all numbers expressing geometries, dimensions, and so forth used in the specification and claims are to be understood at the very least, and not as an attempt to limit the application of the doctrine of equivalents to the scope of the claims, to be construed in light of the number of significant digits and ordinary rounding approaches.

Unless defined otherwise, all technical and scientific terms used herein have the same meanings as commonly understood by one of skill in the art to which the disclosed invention belongs. Publications cited herein and the materials for which they are cited are specifically incorporated by reference.

1. A method for delivering an active agent to a target tissue in a subject, the method comprising:
(i) contacting the target tissue with a Click Target defined by Formula I
X-L¹-CM¹ Formula I
wherein
X represents a tissue binding moiety;
L¹ is absent, or represents a linking group; and
CM¹ represents a first click motif; and
(ii) contacting the target tissue with a Click Prodrug defined by Formula II
A-L²-CM² Formula II
wherein
A represents an active agent;
L¹ is absent, or represents a linking group; and
CM² represents a second click motif complementary to the first click motif;
wherein the first click motif is capable of chemically reacting with the second click motif to form a covalent bond.

2. The method of claim 1, wherein the tissue binding moiety comprises an antibody.

3. The method of claim 1, wherein the tissue binding moiety comprises a lipid which inserts into a cell membrane.

4. The method of claim 1, wherein the tissue binding moiety comprises a functional group capable of chemically reacting with a functional group in a peptide to form a covalent bond.

5. The method of claim 4, wherein the tissue binding moiety comprises a functional group capable of chemically reacting with an amine group in a peptide to form a covalent bond.

6. The method of claim 5, wherein the tissue binding moiety comprises a sulfo-hydroxysuccinimidyl (sNHS) group.

7. The method of claim 1, wherein the first click motif comprises a tetrazine (Tz) and the second click motif comprises an alkene.

8. The method of claim 7, wherein the alkene comprises a cyclooctene.

9. (canceled)

10. The method of claim 1, wherein the first click motif comprises an azide and the second click motif comprises an alkyne.

11. The method of claim 10, wherein the alkyne comprises a cyclooctyne.

12. (canceled)

13. The method of claim 1, wherein L¹ is absent, and L² represents a cleavable linker, such as a hydrolytically cleavable linker, a photocleavable linker, an enzymatically cleavable linker, or a click-cleavable linker.

14. The method of claim 1, wherein the active agent comprises a diagnostic agent.

15. The method of claim 1, wherein the active agent comprises a therapeutic agent.

16. The method of claim 15, wherein the therapeutic agent comprises an anti-cancer drug, a drug that promotes wound healing, a drug that promotes vascularization, a drug that treats or prevents infection, a drug that prevent restenosis, a drug that reduces macular degeneration, a drug that prevents immunological rejection, a drug that prevents thrombosis, or a drug that treats inflammation.

17. (canceled)

18. (canceled)

19. (canceled)

20. (canceled)

21. The method of claim 1, wherein the target tissue comprises a solid tumor.

22. The method of claim 1, wherein contacting the target tissue with a Click Target comprises injecting a pharmaceutical composition comprising the Click Target into the target tissue.

23. The method of claim 1, wherein contacting the target tissue with a Click Prodrug comprises systemically administering the Click Prodrug to the subject.

24. The method of claim 1, further comprising contacting the target tissue with a second Click Prodrug defined by Formula II
A-L²-CM² Formula II
wherein
A represents an active agent;
L¹ is absent, or represents a linking group; and
CM² represents a second click motif complementary to the first click motif.

25. The method of claim 24, wherein the target tissue is contacted with the second Click Prodrug at least one week after the target tissue is contacted with the first Click Prodrug.

26. The method of claim 24, wherein the active agent in the second Click Prodrug is different than the active agent in the first Click Prodrug.

27. A method of maintaining or reducing the size of a tumor in a subject in need thereof, comprising the steps of:
injecting into the tumor a Click Target defined by Formula I
X-L¹-CM¹ Formula I
wherein
X represents a tissue binding moiety;
L¹ is absent, or represents a linking group; and
CM¹ represents a first click motif;
(ii) administering to the subject a Click Prodrug defined by Formula II
A-L²-CM² Formula II
wherein
A represents an anti-cancer drug;
L¹ is absent, or represents a linking group; and
CM² represents a second click motif complementary to the first click motif;
wherein the first click motif is capable of chemically reacting with the second click motif to form a covalent bond; and
(iii) optionally repeating step (ii)
thereby maintaining or reducing the size of the tumor in the subject.

28. (canceled)

29. A method of treating a tumor in a subject, the method comprising
(i) surgically resecting the tumor or a portion thereof;
(ii) contacting tissue surrounding the resected tumor with a Click Target defined by Formula I
X-L¹-CM¹ Formula I
wherein
X represents a tissue binding moiety;
L¹ is absent, or represents a linking group; and
CM¹ represents a first click motif;
(ii) administering to the subject a Click Prodrug defined by Formula II
A-L²-CM² Formula II
wherein
A represents an anti-cancer drug;
L¹ is absent, or represents a linking group; and
CM² represents a second click motif complementary to the first click motif;
wherein the first click motif is capable of chemically reacting with the second click motif to form a covalent bond; and
(iii) optionally repeating step (ii)
thereby maintaining or reducing the size of the tumor in the subject.

30. (canceled)

31. (canceled)
